(19) United States
(12) Patent Application Publication
(10) Pub. No.: US 2006/0276394 A1
(43) Pub. Date: Dec. 7, 2006

(54) METHODS AND COMPOSITIONS FOR TREATING NEUROLOGICAL DISORDERS

(75) Inventors: Christopher William Aston, Brooklyn, NY (US); Jessica Eden Malberg, Robbinsville, NJ (US); Xavier Zafar Khawaja, Yardley, PA (US); Sharon Rosenzweig-Lipson, East Brunswick, NJ (US)

Correspondence Address: PILLSBURY WINTHROP SHAW PITTMAN, LLP, P.O. BOX 10500, MCLEAN, VA, 22102, US

(73) Assignee: WYETH, Madison, NJ (US)

(21) Appl. No.: 11/381,248

(22) Filed: May 2, 2006

Related U.S. Application Data
(63) Continuation-in-part of application No. 10/706,791, filed on Nov. 12, 2003.
(60) Provisional application No. 60/426,472, filed on Nov. 14, 2002.

(51) Int. Cl. A61K 48/00; A61K 31/4704; A61K 38/30

(52) U.S. Cl. 514/12; 514/44; 435/6; 514/310

(57) ABSTRACT

The present invention relates generally to the fields of neuroscience, growth factors and depression. More particularly, the present invention addresses the need in the art for methods and compositions for treating neurological disorders such as depression, anxiety, panic disorder, bi-polar disorder, insomnia, obsessive compulsive disorder, dysthymic disorder and schizophrenia. In certain embodiments, the invention relates to non-covalent binding interactions between insulin-like growth factors (IGFs) and IGF binding proteins (IGFBPs).

### RELATED APPLICATIONS

This is a continuation-in-part of U.S. application Ser. No. 10/706,791, filed on Nov. 12, 2003, which claims priority from U.S. Provisional Application No. 60/426,472, filed Nov. 14, 2002, both of which are incorporated herein in their entirety.

### FIELD OF THE INVENTION

The present invention relates generally to the fields of neuroscience, growth factors and depression. More particularly, the invention relates to insulin-like growth factors (IGFs), insulin-like growth factor binding proteins (IGFBPs) and the role of these proteins in depression, neurogenesis, anxiety and the like.

### BACKGROUND OF THE INVENTION

Insulin-like growth factors (IGFs), which include IGF-I and IGF-II, are involved in a wide array of cellular processes such as proliferation, differentiation and prevention of apoptosis. IGF-I and IGF-II are produced in almost all sites in the body. IGF-I and IGF-II each has its own receptor, but IGF-II will also bind to the IGF-I receptor. The receptors for IGF-I and IGF-II are receptor tyrosine kinases, which signal through the phosphatidyl inositide 3 kinase (PI-3K) and protein kinase B/Akt pathway. IGFs can act in an endocrine manner, a paracrine manner, very close to its site of synthesis in a juxtacrine manner, or on the cells that produce it in an autocrine manner.

IGF-I is the more abundant IGF in serum. In blood and interstitial fluids, free IGF concentration is exceedingly low because the majority of serum IGF is associated with IGF binding proteins (IGFBP). There are seven related members in the IGFBP family (IGFBP-1 to 7). IGFBP-3 is the most abundant member in serum. In serum, IGF-I usually exists as a ternary complex composed of IGF-I (˜7.5 kDa), IGFBP-3 (˜53 kDa) or IGFBP-5, and an acid labile subunit (ALS; ˜150 kDa). The serum half-life of free IGF-I is 10 minutes, the complex of IGF-I and IGFBP-3 is 30 minutes and the ternary complex is about 15 hours.

Thus, IGFBPs generally serve to increase the biological half-life of IGFs and decrease their bioavailability. In some cases however, IGFBPs may potentiate IGF bioactivity, possibly by enhancing interaction of IGFs with the IGF-I receptor (Aston et al., 1996; Bondy and Lee, 1993; Duan and Clemmons, 1998). For example, in vascular smooth muscle cells, IGFBP-5 potentiates the effect of IGF-I (Duan and Clemmons 1998). Despite their common property to interact with IGFs, every IGFBP is expressed in a tightly regulated time-specific and tissue-specific manner, suggesting that each protein may have its own distinct functions.

IGF-I, IGF-II and their receptors are expressed throughout the central nervous system (CNS). Enhanced expression of IGF-I, IGF-II, and IGF receptors occurs in gliomas, meningiomas and other brain tumors. IGF-I mRNA expression is decreased in the hippocampus of aged rats. IGF-II is the most abundantly expressed IGF in the adult CNS (Naeve et al., 2000). IGF-II is able to stimulate proliferation of neuronal and glial cells, and to act as a survival factor for a variety of neuronal cell types. It has been suggested that the main role of IGF-II may be in neuronal regeneration after injury.

IGFBP-1 to 6 are expressed in the CNS. The mRNA expression patterns of IGFBP-2, 4 and 5 in the brain show distinctive non-overlapping distributions (Naeve et al., 2000), suggesting that different IGFBPs perform discrete functions in different parts of the brain.

IGF-II and one of the major CNS binding proteins, IGFBP-2, show a congruency in their anatomical patterns of expression and localization throughout the adult rat brain. Both proteins (i.e., IGF-II and IGFBP-2) are synthesized predominantly in the mesenchymal support structures of the brain, but become localized, remote from the site of synthesis, in the myelin sheaths of individual myelinated axons and in all of the myelinated nerve tracts in the brain, which presumably represents the site of IGF-II bioactivity (Logan et al., 1994). IGF-I, IGFBP-2 and 5 are co-expressed in CNS scar tissue following brain injury. IGFBP-6 preferentially binds IGF-II (Naeve et al., 2000). It is not known whether the ternary complex of IGF-I, IGFBP-3 or 5 and the ALS is found in the brain.

IGF-I is a strong mitogen, inducing proliferation of many cell types including neuronal precursors. In neurons, IGF-I stimulates both neurite outgrowth and proliferation. In Schwann cells, IGF-I increases expression of myelin and stimulates proliferation. Intracerebralventricular IGF-I has been shown to be neuroprotective following hypoxic-ischemic brain injury. Intracerebralventricular IGF-I replacement reverses age-related changes in NMDA receptor subtype and ameliorates the age-related decline in both working and reference memory, and cell proliferation in the dentate gyrus.

Recent studies suggest that IGF-I is able to cross into the cerebrospinal fluid (CSF) (Armstrong et al., 2000; Pulford et al., 2001; Carro et al., 2000). Following subcutaneous deposition of IGF-I in rats, uptake into CSF reached a plateau at plasma concentrations above 150 ng/ml, suggesting carrier-mediated uptake. The efficiency of the process is not high, as concentrations in the CNS were about 0.5% of those in the serum. However, normal concentrations of IGF-I in CSF are 3 ng/ml. It's possible that IGFBPs may have played a role in preventing more IGF from crossing the blood-brain barrier. Neither IGFBPs nor the IGF receptor were required for this uptake, suggesting an alternate carrier system.

Peripheral infusion of IGF-I selectively induces neurogenesis in the dentate gyrus (Aberg et al., 2000), where the IGF-I receptor is expressed (Lesniak et al., 1988; Carro et al., 2000). Lichtenwalner et al., (2001) have demonstrated that intracerebroventricular infusion of IGF-I increases cell proliferation and survival of in the hippocampus. Conversely, blocking the entrance of circulating IGF-I into the brain with a blocking antiserum results in decreased neurogenesis in the dentate gyrus (Trejo et al., 2001).

Transgenic mice overexpressing IGF-I results in an increase in brain size and myelin content (Ye et al., 1995) and increased neurons and synapses in the dentate gyrus (O'Kusky et al., 2000). Conversely, IGF-I knockout mice exhibit a decrease in brain size with fewer hippocampal granule cells (Beck et al., 1995; Cheng et al., 2001). Several transgenic mouse models overexpressing IGFBP-1, 2, 3, and 4 have been developed which have opposing effects. IGFBP-1, 2, and 4 transgenics display lack of somatic growth whereas IGFBP-3 transgenics display organomegaly (Schneider et al., 2000; Hoeflich et al., 2001). Transgenic mice which overexpress IGF-I have increased IGFBP-5 expression in the brain, showing that IGF-I regulates IGFBP-5 expression in the CNS (Ye and D'Ercole, 1998).

Thus, due to their wide range of activities in the CNS, IGF-I and IGF-II have been studied as treatments for a variety of conditions, including amyotrophic lateral sclerosis (commonly known as Lou Gehrig's disease), neuronal regeneration, aging, depression, neurological disorders and the like. Unfortunately, the administration of IGF-I is accompanied by a variety of undesirable side effects, including hypoglycemia, edema (which can cause Bell's palsy, carpal tunnel syndrome, and a variety of other deleterious conditions), hypophosphatemia (low serum phosphorus), and hypernatermia (excessive serum sodium).

Accordingly, there is a need in the art for methods and compositions for administering free IGF-I and/or IGF-II (i.e., unbound, active IGFs) to the CNS, wherein such methods and compositions will be useful in preventing, ameliorating or correcting dysfunctions or diseases related to the CNS.

### SUMMARY OF THE INVENTION

The present invention addresses the need in the art for methods and compositions for treating neurological disorders such as depression, anxiety, panic disorder, bi-polar disorder, insomnia, obsessive compulsive disorder, dysthymic disorder and schizophrenia. More particularly, in certain embodiments, the invention relates to non-covalent binding interactions between insulin-like growth factors (IGFs) and IGF binding proteins (IGFBPs). In certain embodiments, the invention has identified an increase in the expression of insulin-like growth factor binding proteins (IGFBPs), particularly IGFBP-2, in the brains of subjects with major depression. Thus, the present invention, in certain embodiments, is directed to methods for increasing the concentration of unbound IGFs in the CNS via the dissociation of IGF/IGFBP dimeric complex or IGF/IGFBP/ALS trimeric complex, wherein the dissociation of said complex results in an increase in the concentration of free IGF (i.e., unbound, active IGF).

In particular embodiments, the invention is directed to a method for treating a neurological disorder in a human, the method comprising administering to the human a therapeutically effective amount of a composition which dissociates a protein complex comprising an insulin-like growth factor (IGF) and an insulin-like growth factor binding protein (IGFBP). In certain embodiments, the protein complex is further defined as a dimeric complex comprising IGF and IGFBP. In still other embodiments, the protein complex further comprises an acid labile subunit (ALS), wherein the ratio of IGF to IGFBP to ALS is 1:1:1. In yet other embodiments, the composition crosses the blood brain barrier. In certain preferred embodiments, the composition is a small molecule. In certain other embodiments, the composition is a peptide or a peptide mimetic. In still another embodiment, the composition is an antisense molecule which inhibits expression of an IGBFP. In certain other preferred embodiments, the neurological disorder is selected from the group consisting of depression, anxiety, panic disorder, bi-polar disorder, insomnia, obsessive compulsive disorder, dysthymic disorder and schizophrenia. In certain other embodiments, the protein complex is comprised in the central nervous system (CNS). In preferred embodiments, the CNS is defined as the brain, wherein the brain is further defined as a region of the brain selected from the group consisting of the dentate gyrus, the hippocampus the subventricular zone and the cortex. In still another embodiment, the IGFBP is IGFBP-2 or IGFBP-5 and the IGF is IGF-I or IGF-II.

In certain embodiments, the invention is directed to a method of screening for a neurological disorder in a human subject comprising the steps of obtaining a biological sample from the subject, contacting the sample with a polynucleotide probe complementary to an IGFBP-2 mRNA, measuring the amount of probe bound to the mRNA, comparing this amount with IGFBP-2 mRNA in human samples obtained from a statistically significant population lacking the neurological disorder, wherein higher IGFBP-2 levels in the subject indicates a predisposition to the neurological disorder. In particular embodiments, the neurological disorder is selected from the group consisting of depression, anxiety, panic disorder, bipolar disorder, insomnia, obsessive compulsive disorder, dysthymic disorder and schizophrenia. In other embodiments, the biological sample is obtained as a blood sample, a saliva sample, a skin biopsy or a buccal biopsy. In still other embodiments, the biological sample is selected from the group consisting of blood plasma, serum, erythrocytes, leukocytes, platelets, lymphocytes, macrophages, fibroblast cells, mast cells, fat cells and epithelial cells. In one particular embodiment, the probe comprises a nucleotide sequence which hybridizes under high stringency hybridization conditions with a polynucleotide comprising the nucleotide sequence of SEQ ID NO:8, a fragment thereof or a degenerate variant thereof.

In certain other embodiments, the invention is directed to an antisense RNA molecule which inhibits the expression of an IGFBP. In one preferred embodiment, the RNA molecule is antisense to a polynucleotide having a nucleotide sequence of SEQ ID NO:8, a fragment thereof or a degenerate variant thereof.

In still other embodiments, the invention is directed to a pharmaceutical composition which dissociates a protein complex comprising an insulin-like growth factor (IGF) and an insulin-like growth factor binding protein (IGFBP), wherein the molecule crosses the blood brain barrier. In one embodiment, the protein complex is a dimeric complex comprising IGF and IGFBP. In another embodiment, the protein complex further comprises an acid labile subunit (ALS), wherein the ratio of IGF to IGFBP to ALS is 1:1:1. In still other embodiments, the composition is a small molecule or a peptide.

In certain other embodiments, the invention is directed to a method of screening for compounds which dissociate IGF/IGFBP/ALS trimer complex, the method comprising: (a) providing a sample comprising an IGF polypeptide, an IGFBP polypeptide and an ALS polypeptide, wherein the IGFBP is labeled with a radioactive isotope and the IGF is labeled with a scintillant: (b) contacting the sample with a test compound; and (c) detecting light emission of the scintillant, wherein a reduction in light emission, relative to a sample in the absence of the test compound, indicates a test compound which dissociates the complex.

In yet another embodiment, the invention is directed to a method of screening for compounds which dissociate an IGF/IGFBP/ALS trimer complex, the method comprising: (a) providing a sample comprising an IGF polypeptide, an IGFBP polypeptide and an ALS polypeptide, wherein the IGFBP is labeled with a fluorescence donor molecule and the IGF is labeled with a fluorescence acceptor molecule: (b) contacting the sample with a test compound: (c) exciting the sample at the excitation wavelength of the acceptor molecule; and (d) detecting fluorescence at the emission wavelength of the acceptor molecule, wherein a fluorescent signal, relative to a sample in the absence of the test compound, indicates a test compound which dissociates the complex.

In further embodiments, the invention is directed to a method of screening for compounds which dissociate IGF/IGFBP/ALS trimer complex, the method comprising: (a) providing a sample comprising an IGF polypeptide, an IGFBP polypeptide and an ALS polypeptide, wherein the IGF is labeled with a fluorophore: (b) contacting the sample with a test compound: (c) exciting the fluorophore at its excitation wavelength; and (d) detecting the fluorescence polarization of fluorophore, wherein a decrease in polarization, relative to a sample in the absence of the test compound, indicates a test compound which dissociates the complex.

Also provided are methods for treating anxiety or depression in a subject by administering an agent that results in the activation of IGF-1R. For example, the method can comprise administering an agent that elevated IGF-1 levels in brain.

Methods are also provided for treating anxiety or depression in a subject by administering an effective amount of an IGFBP inhibitor. Still further are provided for elevating IGF-1 levels in the brain of a subject by administering an effective amount of an IGFBP inhibitor.

IGFBP inhibitors of the invention are capable of disrupting the interaction of IGF and IGFBP complexes, which may also include an acid labile subunit (ALS). The IGF may be IGF-1 or IGF-2.

For effectiveness in brain, an IGF-1 or IGFBP inhibitor of the invention may be capable of crossing the blood-brain barrier. Alternatively, an IGF-1 or IGFBP inhibitor may be delivered directly to the central nervous system, for example, by intraventricular or intrathecal administration, or other means for direct brain administration.

Representative IGFBP inhibitors that may be used in the disclosed methods include nonpeptide small molecules, peptides and mimetics thereof, and antisense molecules that inhibit IGFBP expression. In a particular aspect of the invention, isoquinoline analogues such as NBI-31772 are used to inhibit IGFBP.

Other features and advantages of the invention will be apparent from the following detailed description, from the preferred embodiments thereof, and from the claims.

### BRIEF DESCRIPTION OF THE DRAWINGS

FIG. 1 demonstrates that IGFBP-5 mRNA is expressed in the dentate gyrus of the mouse hippocampus.

FIG. 2 shows increased expression of IGFBP-2 mRNA in fibroblasts from depressed subjects.

FIG. 3 shows a slight increase in IGFBP-2 mRNA expression in brain tissue from depressed subjects.

FIG. 4 shows enhanced IGF-1 mRNA-1 mRNA expression in antidepressant drug treated C6 glioma cell lines.

FIG. 5 shows enhanced IGF-IA precursor protein expression in antidepressant drug treated rat hippocampus.

FIG. 6 shows differential expression of IGFBP-2 mRNA in anxiolytic drug-treated rat amygdala.

FIG. 7 is a schematic presenting a role for IGFs in depression.

FIG. 8 shows dose-dependent inhibition of ¹²⁵I IGF-I binding to IGFBP-1 to IGFBP-6 by IGF-I and NBI-31772.

FIG. 9 shows homologies of human IGFBPs 1 to 7.

FIG. 10 shows that chronic intracerebroventricular administration of IGF-1 increases proliferation in the adult rat dentate gyrus.

FIG. 11 is a bar graph showing that the IGFBP inhibitor NBI-31772 produced anxiolytic-like effects in the elevated zero maze (EZM). Asterisks, p<0.05 compared to vehicle (VEH) treated group, n=10 animals per group.

FIG. 12 is a bar graph showing that the IGFBP inhibitor NBI-31772 produced anxiolytic-like effects in the four-plate test. Asterisks, p<0.05 compared to vehicle (VEH) treated group, n=10 animals per group.

FIG. 13 is a bar graph showing that the IGF-1 (0.1 μg) produced anxiolytic-like effects in the four-plate test. Asterisk, p<0.05 compared to vehicle (VEH) treated group, n=10 animals per group.

FIG. 14 is a bar graph showing that the IGF-1R inhibitor JB1 (30 μg) blocked the anxiolytic-like effects of IGF-1 (0.1 μg) in the four-plate test. Asterisk, p<0.05 compared to vehicle (VEH) treated group, n=10 animals per group.

FIG. 15 is a bar graph showing that the IGF-1R inhibitor JB1 (10 μg) blocked the anxiolytic-like effects of NBI-31772 (30 μg) in the four-plate test. Asterisk, p<0.05 compared to vehicle (VEH) treated group, n=10 animals per group.

FIG. 16 is a bar graph showing that the IGFBP inhibitor NBI-31772 produced antidepressant-like effects in the tail suspension test. Asterisks, p<0.05 compared to vehicle (VEH) treated group, n=10 animals per group.

FIG. 17 is a bar graph showing that the IGFBP inhibitor IGF-1 produced antidepressant-like effects in the tail suspension test. Asterisk, p<0.05 compared to vehicle (VEH) treated group, n=10 animals per group.

### DETAILED DESCRIPTION OF THE INVENTION

The present invention addresses the need in the art for methods and compositions for treating neurological disorders such as depression, anxiety, panic disorder, bi-polar, insomnia, obsessive compulsive disorder, dysthymic disorder and schizophrenia. More particularly, in certain embodiments, the invention relates to disrupting non-covalent binding interactions between insulin-like growth factors (IGFs) and IGF binding proteins (IGFBPs). IGFs, which include IGF-I and IGF-II, are involved in a wide array of cellular processes such as neuron proliferation, neuron differentiation and prevention of apoptosis. For example, free IGF-II (i.e., unbound, active IGF) is able to stimulate proliferation of neuronal and glial cells. However, IGFBP-2, the major binding protein for IGF-II in the central nervous system (CNS), associates with (i.e., binds) IGF-II, thereby decreasing IGF-II bioavailability. Thus, it is highly desirable to identify methods and compositions which dissociate an IGF from its IGFBP binding partner, therein effectively increasing free IGF concentrations in vivo.

As defined hereinafter, the terms “free IGF”, “unbound IGF” and “active IGF” may be used interchangeably, wherein an “active IGF” is an IGF polypeptide which can bind with its IGF receptor. Similarly, as defined hereinafter, the terms “bound IGF”, “associated IGF” and “inactive IGF” may be used interchangeably, wherein “bound IGF” is at least a dimeric complex comprising an IGF and an IGFBP (e.g., IGF/IGFBP), wherein “bound IGF” (IGF/IGFBP) has a reduced or null ability to bind to its IGF receptor, relative to “active IGF”. As defined hereinafter, a dimeric complex of “IGF and IGFBP” is represented by the formula “IGF/IGFBP” and a trimeric complex of “IGF, IGFBP and an acid labile subunit (hereinafter, ALS)” is represented by the formula “IGF/IGFBP/ALS”.

In certain embodiments, the invention has identified an increase in the expression of IGFBPs, particularly IGFBP-2, in the brains of subjects with major depression. Thus, the present invention, in particular embodiments, is directed to methods for increasing the concentration of active IGF in the CNS via the dissociation of IGF/IGFBP dimeric complex or IGF/IGFBP/ALS trimeric complex, wherein the dissociation of said complex results in an increase in the concentration of free IGF (i.e., unbound, active IGF). As defined hereinafter, a compound or a composition which “dissociates” an IGF/IGFBP dimer or an IGF/IGFBP/ALS trimer may be any molecule that can disrupt non-covalent interactions of the dimer or trimer, wherein the disruption of non-covalent interactions results in active IGF monomers. As defined hereinafter, a human “IGF” polypeptide includes IGF-I and IGF-II, unless otherwise stated. As defined hereinafter, a human “IGF-I” polypeptide may exist as either of its alternately spliced forms, refered to herein as “IGF-IA” (SEQ ID NO:2) and “IGF-IB” (SEQ ID NO:3). As defined hereinafter, a human “IGFBP” includes IGFBP-1 to IGFBP-7, unless otherwise stated.

A. IGF, IGFBP and ALS Polypeptides

In certain embodiments, the invention is directed to methods for screening compounds which dissociate an IGF/IGFBP dimer complex or an IGF/IGFBP/ALS trimer complex. In other embodiments, the invention is directed to peptides or peptide mimetics which dissociate IGF/IGFBP dimer complex or IGF/IGFBP/ALS trimer complex.

Thus, in particular embodiments, the present invention provides isolated and purified IGF, IGFBP and ALS polypeptides, or fragments thereof. Preferably, a full length polypeptide of the invention is a recombinant polypeptide. Typically, an IGF, IGFBP or ALS polypeptide is produced by recombinant expression in a non-human cell. IGF, IGFBP and ALS polypeptide fragments of the invention may be recombinantly expressed or prepared via peptide synthesis methods known in the art (Barany et al., 1987; U.S. Pat. No. 5,258,454).

Human IGF-I polypeptide is expressed in vivo as IGF-IA or IGF-IB (i.e., alternately spliced IGF-I). Thus, the amino acid sequence of human IGF-IA polypeptide is represented as SEQ ID NO:2 and the amino acid sequence of human IGF-IB polypeptide is represented as SEQ ID NO:3. The amino acid sequence of human IGF-II polypeptide is represented as SEQ:4. The amino acid sequences of human IGFBP-1, IGFBP-2, IGFBP-3, IGFBP-4, IGFBP-6 and IGFBP-7 polypeptides are represented as SEQ ID NO:7, SEQ ID NO:9, SEQ ID NO:11, SEQ ID NO:13, SEQ ID NO:15, SEQ ID NO:17 and SEQ ID NO:19, respectively. The amino acid sequence of human ALS polypeptide is represented as SEQ ID NO:21.

An IGF or IGFBP polypeptide of the invention includes any functional variants of a human IGF or IGFBP polypeptide. Functional allelic variants are naturally occurring amino acid sequence variants of a human IGF polypeptide or IGFBP polypeptide that maintain the ability to bind an IGF receptor or bind an IGF polypeptide, respectively. Functional allelic variants will typically contain only conservative substitution of one or more amino acids, or substitution, deletion or insertion of non-critical residues in non-critical regions of the polypeptide.

Modifications and changes can be made in the structure of a polypeptide of the present invention and still obtain a molecule having IGF, IGFBP or ALS characteristics. For example, certain amino acids can be substituted for other amino acids in a sequence without appreciable loss of receptor activity. Because it is the interactive capacity and nature of a polypeptide that defines that polypeptide's biological functional activity, certain amino acid sequence substitutions can be made in a polypeptide sequence (or, of course, its underlying DNA coding sequence) and nevertheless obtain a polypeptide with like properties.

In making such changes, the hydropathic index of amino acids can be considered. The importance of the hydropathic amino acid index in conferring interactive biologic function on a polypeptide is generally understood in the art (Kyte & Doolittle, 1982). It is known that certain amino acids can be substituted for other amino acids having a similar hydropathic index, or score, and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid residue determines the secondary and tertiary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within +/−2 is preferred, those which are within +/−1 are particularly preferred, and those within +/−0.5 are even more particularly preferred.

Substitution of like amino acids can also be made on the basis of hydrophilicity, particularly where the biological functional equivalent polypeptide, or peptide thereby created, is intended for use in immunological embodiments. U.S. Pat. No. 4,554,101, incorporated by reference herein in its entirety, states that the greatest local average hydrophilicity of a polypeptide, as governed by the hydrophilicity of its adjacent amino acids, correlates with its immunogenicity and antigenicity, i.e. with a biological property of the polypeptide.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those which are within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take of the foregoing various characteristics into consideration are well known to those of skill in the art and include: arginine and lysine; glutamate and aspartate; serine and threonine; glutamine and asparagine; and valine, leucine and isoleucine (see Table 1 below). The present invention thus contemplates functional or biological equivalents of a polypeptide as set forth above.

TABLE 1
EXEMPLARY AMINO ACID SUBSTITUTIONS

| Original Residue | Exemplary Residue Substitution |
|---|---|
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Leu; Tyr |
| Ser | Thr |
| Thr | Ser |
| Trp | Tyr |
| Tyr | Trp; Phe |
| Val | Ile; Leu |

Biological or functional equivalents of a polypeptide can also be prepared using site-specific mutagenesis. Site-specific mutagenesis is a technique useful in the preparation of second generation polypeptides, or biologically functional equivalent polypeptides or peptides, derived from the sequences thereof, through specific mutagenesis of the underlying DNA. As noted above, such changes can be desirable where amino acid substitutions are desirable. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

It is contemplated in the present invention, that an IGF polypeptide or an IGFBP polypeptide may advantageously be cleaved into fragments for use in further structural or functional analysis, or in the generation of reagents such as IGF or IGFBP-related polypeptides and IGF or IGFBP-specific antibodies. This can be accomplished by treating purified or unpurified polypeptide with a protease such as glu-C (Boehringer, Indianapolis, Ind.), trypsin, chymotrypsin, V8 protease, pepsin and the like. Treatment with CNBr is another method by which IGF or IGFBP fragments may be produced from natural IGF or IGFBP. Recombinant techniques also can be used to express specific fragments (e.g., an IGF-IGFBP binding domain) of an IGF polypeptide. In one example, the invention provides an IGF polypeptide fragment which binds an IGFBP polypeptide. It is contemplated that such an IGF fragment may be engineered to be a high affinity ligand for IGFBP, wherein the IGF fragment competes with and/or displaces a full length IGF polypeptide at the IGF binding site of the IGFBP polypeptide.

In addition, the invention also contemplates that compounds sterically similar to IGF may be formulated to mimic the key portions of the peptide structure, called peptidomimetics or peptide mimetics. Mimetics are peptide-containing molecules which mimic elements of polypeptide secondary structure. See, for example, Johnson et al. (1993); and U.S. Pat. No. 5,817,879. The underlying rationale behind the use of peptide mimetics is that the peptide backbone of polypeptides exists chiefly to orient amino acid side chains in such a way as to facilitate molecular interactions, such as those of receptor and ligand.

Successful applications of the peptide mimetic concept have thus far focused on mimetics of β-turns within polypeptides. Likely β-turn structures within an IGF polypeptide can be predicted by computer-based algorithms. U.S. Pat. No. 5,933,819 describes a neural network based method and system for identifying relative peptide binding motifs from limited experimental data. In particular, an artificial neural network (ANN) is trained with peptides with known sequences and function (i.e., binding strength) identified from a phage display library. The ANN is then challenged with unknown peptides and predicts relative binding motifs. Analysis of the unknown peptides validate the predictive capability of the ANN. Once the component amino acids of the turn are determined, mimetics can be constructed to achieve a similar spatial orientation of the essential elements of the amino acid side chains, as discussed in Johnson et al. (1993); U.S. Pat. No. 6,420,119 and U.S. Pat. No. 5,817,879.

B. Isolated Polynucleotides

In certain embodiments, the invention is directed to methods of screening for a neurological disorder in humans comprising the steps of obtaining a biological sample from the subject, contacting the sample with a polynucleotide probe complementary to an IGFBP mRNA, measuring the amount of probe bound to the mRNA, comparing this amount with IGFBP mRNA in human samples obtained from a statistically significant population lacking the neurological disorder, wherein higher IGFBP levels in the subject indicates a predisposition to the neurological disorder. In other embodiments, the invention is directed to antisense polynucleotide or antisense oligonucleotide molecules, wherein the antisense molecules are used to inhibit the expression of an IGFBP. In still other embodiments, IGF, IGBFP and ALS polypeptides, or fragments thereof, are recombinantly expressed.

Thus, in one aspect, the present invention provides isolated and purified polynucleotides that encode IGF, IGFBP and ALS polypeptides. In particular embodiments, a polynucleotide of the present invention is a DNA molecule.

Due to the degeneracy of the genetic code, an IGF-I polynucleotide of the invention is any polynucleotide encoding an IGF-I polypeptide having at least about 80%, more preferably about 90% and even more preferably about 95% sequence identity to an IGF-I polypeptide of SEQ ID NO:2 or SEQ ID NO:3. Similarly, an IGF-II polynucleotide of the invention is any polynucleotide encoding an IGF-II polypeptide having at least about 80%, more preferably at least about 90% and even more preferably at least about 95% sequence identity to an IGF-II polypeptide of SEQ ID NO:4. An IGFBP polynucleotide of the invention is any polynucleotide encoding an IGFBP polypeptide having at least about 80%, more preferably at least about 90% and even more preferably at least about 95% sequence identity to an IGFBP polypeptide having an amino acid sequence of SEQ ID NO:7, SEQ ID NO:9, SEQ ID NO:11, SEQ ID NO:13, SEQ ID NO:15, SEQ ID NO:17 or SEQ ID NO:19. An ALS polynucleotide of the invention is any polynucleotide encoding an ALS polypeptide having at least about 80%, more preferably at least about 90% and even more preferably at least about 95% sequence identity to an ALS polypeptide of SEQ ID NO:21.

An isolated polynucleotide encoding an IGF-I polypeptide of SEQ ID NO:2 (IGF-IA) and SEQ ID NO:3 (IGF-IB) has a nucleotide sequence shown in SEQ ID NO:1. An isolated polynucleotide encoding an IGF-II polypeptide of SEQ ID NO:5 has a nucleotide sequence shown in SEQ ID NO:4. An isolated polynucleotide encoding an IGFBP-1 polypeptide of SEQ ID NO:7, an IGFBP-2 polypeptide SEQ ID NO:9, an IGFBP-3 polypeptide of SEQ ID NO:11, an IGFBP-4 polypeptide of SEQ ID NO:13, an IGFBP-5 polypeptide of SEQ ID NO:15, an IGFBP-6 polypeptide of SEQ ID NO:17 and an IGFBP-7 polypeptide of SEQ ID NO:19 has a nucleotide sequence shown in SEQ ID NO:6, SEQ ID NO:8, SEQ ID NO:10, SEQ ID NO:12, SEQ ID NO:14, SEQ ID NO:16 and SEQ ID NO:18, respectively. An isolated polynucleotide encoding an ALS polypeptide of SEQ ID NO:21 has a nucleotide sequence shown in SEQ ID NO:20.

As used herein, the term “polynucleotide” means a sequence of nucleotides connected by phosphodiester linkages. Polynucleotides are presented herein in the direction from the 5′ to the 3′ direction. A polynucleotide of the present invention can comprise from about 40 to about several hundred thousand base pairs. Preferably, a polynucleotide comprises from about 10 to about 3,000 base pairs. Preferred lengths of particular polynucleotide are set forth hereinafter.

A polynucleotide of the present invention can be a deoxyribonucleic acid (DNA) molecule, a ribonucleic acid (RNA) molecule, or analogs of the DNA or RNA generated using nucleotide analogs. The nucleic acid molecule can be single-stranded or double-stranded, but preferably is double-stranded DNA. Where a polynucleotide is a DNA molecule, that molecule can be a gene, a cDNA molecule or a genomic DNA molecule. Nucleotide bases are indicated herein by a single letter code: adenine (A), guanine (G), thymine (T), cytosine (C), inosine (I) and uracil (U).

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated,” as the term is employed herein.

Polynucleotides of the present invention may be obtained, using standard cloning and screening techniques, from a cDNA library derived from mRNA from human cells or from genomic DNA. Polynucleotides of the invention can also synthesized using well known and commercially available techniques.

In another preferred embodiment, an isolated polynucleotide of the invention comprises a nucleic acid molecule which is a complement of the nucleotide sequence shown in SEQ ID NO:1, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, or a fragment of one of these nucleotide sequences. A nucleic acid molecule which is complementary to the nucleotide sequence shown in SEQ ID NO:1, SEQ ID NO:4, SEQ ID NO:6, SEQ ID NO:8, SEQ ID NO:10, SEQ ID NO:12, SEQ ID NO:14, SEQ ID NO:16, SEQ ID NO:18 or SEQ ID NO:20 is one which is sufficiently complementary to the nucleotide sequence, such that it can hybridize to the nucleotide sequence shown in SEQ ID NO:1, SEQ ID NO:4, SEQ ID NO:6, SEQ ID NO:8, SEQ ID NO:10, SEQ ID NO:12, SEQ ID NO:14, SEQ ID NO:16, SEQ ID NO:18 or SEQ ID NO:20, thereby forming a stable duplex. Examples of hybridization stringency conditions are detailed in Table 2.

Moreover, the polynucleotide of the invention can comprise only a fragment of the coding region of a polynucleotide or gene, such as a fragment of SEQ ID NO:1, SEQ ID NO:4, SEQ ID NO:6, SEQ ID NO:8, SEQ ID NO:10, SEQ ID NO:12, SEQ ID NO:14, SEQ ID NO:16, SEQ ID NO:18 or SEQ ID NO:20.

When the polynucleotides of the invention are used for the recombinant production of IGF, IGFBP and ALS polypeptides of the present invention, the polynucleotide may include the coding sequence for the mature polypeptide, by itself, or the coding sequence for the mature polypeptide in reading frame with other coding sequences, such as those encoding a leader or secretory sequence, a pre-, or pro- or prepro-polypeptide sequence, or other fusion peptide portions. For example, a marker sequence which facilitates purification of the fused polypeptide can be encoded (see Gentz et al., 1989, incorporated herein by reference). The polynucleotide may also contain non-coding 5′ and 3′ sequences, such as transcribed, non-translated sequences, splicing and polyadenylation signals, ribosome binding sites and sequences that stabilize mRNA.

As used herein, the terms “gene” and “recombinant gene” refer to polynucleotides comprising an open reading frame encoding an IGF, IGFBP or ALS polypeptide, preferably a human polypeptide.

In certain embodiments, the polynucleotide sequence information provided by the present invention allows for the preparation of relatively short DNA (or RNA) oligonucleotide sequences having the ability to specifically hybridize to gene sequences of the selected polynucleotides disclosed herein. In a preferred embodiment, an oligonucleotide sequence is one which is complimentary to an IGFBP-2 mRNA. The term “oligonucleotide” as used herein is defined as a molecule comprised of two or more deoxyribonucleotides or ribonucleotides, usually more than three (3), and typically more than ten (10) and up to one hundred (100) or more (although preferably between twenty and thirty). The exact size will depend on many factors, which in turn depends on the ultimate function or use of the oligonucleotide. Thus, in particular embodiments of the invention, nucleic acid probes of an appropriate length are prepared based on a consideration of a selected nucleotide sequence, e.g., a sequence such as that shown in SEQ ID NO:1, SEQ ID NO:4, SEQ ID NO:6, SEQ ID NO:8, SEQ ID NO:10, SEQ ID NO:12, SEQ ID NO:14, SEQ ID NO:16, SEQ ID NO:18 or SEQ ID NO:20. The ability of such nucleic acid probes to specifically hybridize to a polynucleotide encoding an IGFBP lends them particular utility in a variety of embodiments. Most importantly, the probes can be used in a variety of assays for detecting the presence of complementary sequences in a given sample.

In certain embodiments, it is advantageous to use oligonucleotide primers. These primers may be generated in any manner, including chemical synthesis, DNA replication, reverse transcription, or a combination thereof. The sequence of such primers is designed using a polynucleotide of the present invention for use in detecting, amplifying or mutating a defined segment of a gene or polynucleotide that encodes a polypeptide from mammalian cells using polymerase chain reaction (PCR) technology.

In certain embodiments, it is advantageous to employ a polynucleotide of the present invention in combination with an appropriate label for detecting hybrid formation. A wide variety of appropriate labels are known in the art, including radioactive, enzymatic or other ligands, such as avidin/biotin, which are capable of giving a detectable signal.

Polynucleotides which are identical or sufficiently identical to a nucleotide sequence contained in SEQ ID NO:1, SEQ ID NO:4, SEQ ID NO:6, SEQ ID NO:8, SEQ ID NO:10, SEQ ID NO:12, SEQ ID NO:14, SEQ ID NO:16, SEQ ID NO:18 or SEQ ID NO:20 or a fragment thereof, may be used as hybridization probes for cDNA and genomic DNA or as primers for a nucleic acid amplification (PCR) reaction, to isolate full-length cDNAs and genomic clones encoding polypeptides of the present invention and to isolate cDNA and genomic clones of other genes (including genes encoding homologs and orthologs from species other than mouse) that have a high sequence similarity to SEQ ID NO:1, SEQ ID NO:4, SEQ ID NO:6, SEQ ID NO:8, SEQ ID NO:10, SEQ ID NO:12, SEQ ID NO:14, SEQ ID NO:16, SEQ ID NO:18 or SEQ ID NO:20 or a fragment thereof. Typically these nucleotide sequences are from at least about 70% identical to at least about 95% identical to that of the reference polynucleotide sequence. The probes or primers will generally comprise at least 15 nucleotides, preferably, at least 30 nucleotides and may have at least 50 nucleotides. Particularly preferred probes will have between 30 and 50 nucleotides.

There are several methods available and well known to those skilled in the art to obtain full-length cDNAs, or extend short cDNAs, for example those based on the method of Rapid Amplification of cDNA ends (RACE) (see, Frohman et al., 1988). Recent modifications of the technique, exemplified by the Marathon™ technology (Clontech Laboratories Inc.) for example, have significantly simplified the search for longer cDNAs. In the Marathon™ technology, cDNAs have been prepared from mRNA extracted from a chosen tissue and an “adaptor” sequence ligated onto each end. Nucleic acid amplification (PCR) is then carried out to amplify the “missing” 5′ end of the cDNA using a combination of gene specific and adaptor specific oligonucleotide primers. The PCR reaction is then repeated using “nested” primers, that is, primers designed to anneal within the amplified product (typically an adaptor specific primer that anneals further 3′ in the adaptor sequence and a gene specific primer that anneals further 5′ in the known gene sequence). The products of this reaction can then be analyzed by DNA sequencing and a full-length cDNA constructed either by joining the product directly to the existing cDNA to give a complete sequence, or by carrying out a separate full-length PCR using the new sequence information for the design of the 5′ primer.

To provide certain advantages in accordance with the present invention, a preferred nucleic acid sequence employed for hybridization studies or assays includes probe molecules that are complementary to at least a 10 to 70 or so long nucleotide stretch of a polynucleotide that encodes a polypeptide of the invention. A size of at least 10 nucleotides in length helps to ensure that the fragment will be of sufficient length to form a duplex molecule that is both stable and selective. Molecules having complementary sequences over stretches greater than 10 bases in length are generally preferred, though, in order to increase stability and selectivity of the hybrid, and thereby improve the quality and degree of specific hybrid molecules obtained. One will generally prefer to design nucleic acid molecules having gene-complementary stretches of 25 to 40 nucleotides, 55 to 70 nucleotides, or even longer where desired. Such fragments can be readily prepared by, for example, directly synthesizing the fragment by chemical means, by application of nucleic acid reproduction technology, such as the PCR technology of U.S. Pat. No. 4,683,202 (incorporated by reference herein in its entirety) or by excising selected DNA fragments from recombinant plasmids containing appropriate inserts and suitable restriction enzyme sites.

Accordingly, a polynucleotide probe molecule of the invention can be used for its ability to selectively form duplex molecules with complementary stretches of the gene. Depending on the application envisioned, one will desire to employ varying conditions of hybridization to achieve a varying degree of selectivity of the probe toward the target sequence. For applications requiring a high degree of selectivity, one will typically desire to employ relatively stringent conditions to form the hybrids (see Table 2 below).

The present invention also includes polynucleotides capable of hybridizing under reduced stringency conditions, more preferably stringent conditions, and most preferably highly stringent conditions, to polynucleotides described herein. Examples of stringency conditions are shown in Table 2 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 2
HYBRIDIZATION STRINGENCY CONDITIONS

| | Poly- | Hybrid | Hybridization | Wash |
|---|---|---|---|---|
| Stringency | nucleotide | Length | Temperature and | Temperature |
| Condition | Hybrid | (bp)^{I} | Buffer^{H} | and BufferH |
| A | DNA:DNA | >50 | 65° C.; 1 × SSC | 65° C.; |
| | | | -or- 42° C.; 1 × SSC, | 0.3 × SSC |
| | | | 50% formamide | |
| B | DNA:DNA | <50 | T_{B}; 1 × SSC | T_{B}; |
| C | DNA:RNA | >50 | 67° C.; 1 × SSC | 1 × SSC |
| | | | -or- 45° C.; 1 × SSC, | 67° C.; |
| | | | 50% formamide | 0.3 × SSC |
| D | DNA:RNA | <50 | T_{D}; 1 × SSC | T_{D}; |
| E | RNA:RNA | >50 | 70° C.; 1 × SSC | 1 × SSC |
| | | | -or- 50° C.; 1 × SSC, | 70° C.; |
| | | | 50% formamide | 0.3 × SSC |
| F | RNA:RNA | <50 | T_{F}; 1 × SSC | T_{f}; |
| G | DNA:DNA | >50 | 65° C.; 4 × SSC | 1 × SSC |
| | | | -or- 42° C.; 4 × SSC, | 65° C.; |
| | | | 50% formamide | 1 × SSC |
| H | DNA:DNA | <50 | T_{H}; 4 × SSC | T_{H}; |
| I | DNA:RNA | >50 | 67° C.; 4 × SSC | 4 × SSC |
| | | | -or- 45° C.; 4 × SSC, | 67° C.; |
| | | | 50% formamide | 1 × SSC |
| J | DNA:RNA | <50 | T_{J}; 4 × SSC | T_{J}; |
| K | RNA:RNA | >50 | 70° C.; 4 × SSC | 4 × SSC |
| | | | -or- 50° C.; 4 × SSC, | 67° C.; |
| | | | 50% formamide | 1 × SSC |
| L | RNA:RNA | <50 | T_{L}; 2 × SSC | T_{L}; |
| M | DNA:DNA | >50 | 50° C.; 4 × SSC | 2 × SSC |
| | | | -or- 40° C.; 6 × SSC, | 50° C.; |
| | | | 50% formamide | 2 × SSC |
| N | DNA:DNA | <50 | T_{N}; 6 × SSC | T_{N}; |
| O | DNA:RNA | >50 | 55° C.; 4 × SSC | 6 × SSC |
| | | | -or- 42° C.; 6 × SSC, | 55° C.; |
| | | | 50% formamide | 2 × SSC |
| P | DNA:RNA | <50 | T_{P}; 6 × SSC | T_{P}; |
| Q | RNA:RNA | >50 | 60° C.; 4 × SSC | 6 × SSC |
| | | | -or- 45° C.; 6 × SSC, | 60° C.; |
| | | | 50% formamide | 2 × SSC |
| R | RNA:RNA | <50 | T_{R}; 4 × SSC | T_{R}; |
| | | | | 4 × SSC |
| (bp)^{I}: The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length is determined by aligning the sequences of the polynucleotides and identifying the region or regions of optimal sequence complementarity. | | | | |
| Buffer^{H}: SSPE (1 × SSPE is 0.15M NaCl, 10 mM NaH₂PO₄, and 1.25 mM EDTA, pH 7.4) can be substituted for SSC (1 × SSC is 0.15M NaCl and 15 mM sodium citrate) in the hybridization and wash buffers; washes are performed for 15 minutes after hybridization is complete. | | | | |
| T_{B} through T_{R}: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). | | | | |
| # For hybrids between 18 and 49 base pairs in length, Tₘ(° C.) = 81.5 + 16.6(log₁₀[Na⁺]) + 0.41(% G + C) − (600/N), where N is the number of bases in the hybrid, and [Na⁺] is the concentration of sodium ions in the hybridization buffer ([Na⁺] for 1 × SSC = 0.165 M). | | | | |

In addition to the nucleic acid molecules encoding IGF, IGFBP and ALS polypeptides described above, another aspect of the invention pertains to isolated nucleic acid molecules which are antisense to IGFBP. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire IGFBP coding strand (e.g., SEQ ID NO:8), or to only a fragment thereof. In one embodiment, an antisense nucleic acid molecule is antisense to a “coding region” of the coding strand of a nucleotide sequence encoding an IGFBP polypeptide.

The term “coding region” refers to the region of the nucleotide sequence comprising codons which are translated into amino acid residues, e.g., the entire coding region of SEQ ID NO:8. In another embodiment, the antisense nucleic acid molecule is antisense to a “noncoding region” of the coding strand of a nucleotide sequence encoding an IGFBP polypeptide. The term “noncoding region” refers to 5′ and 3′ sequences which flank the coding region that are not translated into amino acids (i.e., also referred to as 5′ and 3′ untranslated regions).

Given the coding strand sequence encoding the IGFBP polypeptide disclosed herein (e.g., SEQ ID NO:8), antisense nucleic acids of the invention can be designed according to the rules of Watson and Crick base pairing. The antisense nucleic acid molecule can be complementary to the entire coding region of IGFBP mRNA, but more preferably is an oligonucleotide which is antisense to only a fragment of the coding or noncoding region of IGFBP mRNA. For example, the antisense oligonucleotide can be complementary to the region surrounding the translation start site of IGFBP mRNA.

An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an IGFBP, preferably an IGFBP-2 polypeptide to thereby inhibit expression of the polypeptide, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of an antisense nucleic acid molecule of the invention includes direct injection at a tissue site. Alternatively, an antisense nucleic acid molecule can be modified to target selected cells and then administered systemically. For example, for systemic administration, an antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual γ-units, the strands run parallel to each other (Gaultier et al., 1987). The antisense nucleic acid molecule can also comprise a 2′-o-methylribonucleotide (Inoue et al., 1987) or a chimeric RNA-DNA analogue (Inoue et al., 1987).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach, 1988)) can be used to catalytically cleave IGFBP mRNA transcripts to thereby inhibit translation of IGFBP mRNA. A ribozyme having specificity for an IGFBP-encoding nucleic acid can be designed based upon the nucleotide sequence of the IGFBP genomic DNA. For example, a derivative of a Tetrahymena L-19 IVS RNA can be constructed in which the nucleotide sequence of the active site is complementary to the nucleotide sequence to be cleaved in an IGFBP-encoding mRNA. See, e.g., Cech et al. U.S. Pat. No. 4,987,071 and Cech et al. U.S. Pat. No. 5,116,742, both of which are incorporated by reference herein in their entirety. Alternatively, IGFBP mRNA can be used to select a catalytic RNA having a specific ribonuclease activity from a pool of RNA molecules. See, e.g., Bartel and Szostak, 1993.

Alternatively, IGFBP gene expression can be inhibited by targeting nucleotide sequences complementary to the regulatory region of the IGFBP gene (e.g., the IGFBP gene promoter and/or enhancers) to form triple helical structures that prevent transcription of the IGFBP gene in target cells. See generally, Helene, 1991; Helene et al., 1992; and Maher, 1992.

IGFBP gene expression can also be inhibited using RNA interference (RNAi). This is a technique for post-transcriptional gene silencing (PTGS), in which target gene activity is specifically abolished with cognate double-stranded RNA (dsRNA). RNAi resembles in many aspects PTGS in plants and has been detected in many invertebrates including trypanosome, hydra, planaria, nematode and fruit fly (Drosophila melanogaster). It may be involved in the modulation of transposable element mobilization and antiviral state formation. RNAi in mammalian systems is disclosed in International Application No. WO 00/63364 which is incorporated by reference herein in its entirety. Basically, dsRNA of at least about 600 nucleotides, homologous to the target (IGFBP) is introduced into the cell and a sequence specific reduction in gene activity is observed.

C. Vectors, Host Cells and Recombinant Polypeptides

In an alternate embodiment, the present invention provides expression vectors comprising polynucleotides that encode IGF, IGFBP or ALS polypeptides. Preferably, the expression vectors of the invention comprise polynucleotides operatively linked to an enhancer-promoter. In certain embodiments, the expression vectors of the invention comprise polynucleotides operatively linked to a prokaryotic promoter. Alternatively, the expression vectors of the present invention comprise polynucleotides operatively linked to an enhancer-promoter that is a eukaryotic promoter, and the expression vectors further comprise a polyadenylation signal that is positioned 3′ of the carboxy-terminal amino acid and within a transcriptional unit of the encoded polypeptide.

Expression of proteins in prokaryotes is most often carried out in E. coli with vectors containing constitutive or inducible promoters directing the expression of either fusion or non-fusion proteins. Fusion vectors add a number of amino acids to a protein encoded therein, to the amino or carboxy terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith and Johnson, 1988), pMAL (New England Biolabs, Beverly; MA) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., 1988) and pET IId (Studier et al., 1990). Target gene expression from the pTrc vector relies on host RNA polymerase transcription from a hybrid trp-lac fusion promoter. Target gene expression from the pET IId vector relies on transcription from a T7 gn1 β-lac fusion promoter mediated by a coexpressed viral RNA polymerase T7 gnl. This viral polymerase is supplied by host strains BL21 (DE3) or HMS 174(DE3) from a resident prophage harboring a T7 gnl gene under the transcriptional control of the lacUV 5 promoter.

One strategy to maximize recombinant protein expression in E. coli is to express the protein in a host bacteria with an impaired capacity to proteolytically cleave the recombinant protein. Another strategy is to alter the nucleic acid sequence of the nucleic acid to be inserted into an expression vector so that the individual codons for each amino acid are those preferentially utilized in E. coli. Such alteration of nucleic acid sequences of the invention can be carried out by standard DNA mutagenesis or synthesis techniques.

In another embodiment, the polynucleotide expression vector is a yeast expression vector. Examples of vectors for expression in yeast S. cerivisae include pYepSec I (Baldari, et al., 1987), pMFa (Kurjan and Herskowitz, 1982), pJRY88 (Schultz et al., 1987), and pYES2 (Invitrogen Corporation, San Diego, Calif.), p416GPD and p426GPD (Mumberg et al., 1995).

In yet another embodiment, a polynucleotide of the invention is expressed in mammalian cells using a mammalian expression vector. Examples of mammalian expression vectors include pCDM8 (Seed, 1987) and pMT2PC (Kaufman et al., 1987). When used in mammalian cells, the expression vector's control functions are often provided by viral regulatory elements.

For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., “Molecular Cloning: A Laboratory Manual” 2nd, ed, Cold Spring Harbor Laboratory, Cold Spring Harbor Laboratory Press, Cold Spring Harbor, N.Y., 1989, incorporated herein by reference.

A promoter is a region of a DNA molecule typically within about 100 nucleotide pairs in front of (upstream of) the point at which transcription begins (i.e., a transcription start site). That region typically contains several types of DNA sequence elements that are located in similar relative positions in different genes. As used herein, the term “promoter” includes what is referred to in the art as an upstream promoter region, a promoter region or a promoter of a generalized eukaryotic RNA Polymerase II transcription unit.

Another type of discrete transcription regulatory sequence element is an enhancer. An enhancer provides specificity of time, location and expression level for a particular encoding region (e.g., gene). A major function of an enhancer is to increase the level of transcription of a coding sequence in a cell that contains one or more transcription factors that bind to that enhancer. Unlike a promoter, an enhancer can function when located at variable distances from transcription start sites so long as a promoter is present.

As used herein, the phrase “enhancer-promoter” means a composite unit that contains both enhancer and promoter elements. An enhancer-promoter is operatively linked to a coding sequence that encodes at least one gene product. As used herein, the phrase “operatively linked” means that an enhancer-promoter is connected to a coding sequence in such a way that the transcription of that coding sequence is controlled and regulated by that enhancer-promoter. Means for operatively linking an enhancer-promoter to a coding sequence are well known in the art. As is also well known in the art, the precise orientation and location relative to a coding sequence whose transcription is controlled, is dependent inter alia upon the specific nature of the enhancer-promoter. Thus, a TATA box minimal promoter is typically located from about 25 to about 30 base pairs upstream of a transcription initiation site and an upstream promoter element is typically located from about 100 to about 200 base pairs upstream of a transcription initiation site. In contrast, an enhancer can be located downstream from the initiation site and can be at a considerable distance from that site.

A coding sequence of an expression vector is operatively linked to a transcription terminating region. RNA polymerase transcribes an encoding DNA sequence through a site where polyadenylation occurs. Typically, DNA sequences located a few hundred base pairs downstream of the polyadenylation site serve to terminate transcription. Those DNA sequences are referred to herein as transcription-termination regions. Those regions are required for efficient polyadenylation of transcribed messenger RNA (mRNA). Transcription-terminating regions are well known in the art. A preferred transcription-terminating region used in an adenovirus vector construct of the present invention comprises a polyadenylation signal of SV40 or the protamine gene.

The invention further provides a recombinant expression vector comprising a DNA molecule encoding an IGFBP polypeptide cloned into the expression vector in an antisense orientation. That is, the DNA molecule is operatively linked to a regulatory sequence in a manner which allows for expression (by transcription of the DNA molecule) of an RNA molecule which is antisense to IGFBP mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. Listed in Table 3 are non-limiting examples of tissue-specific promoter contemplated for use.

TABLE 3
TISSUE SPECIFIC PROMOTERS

| PROMOTER | Target |
|---|---|
| Tyrosinase | Melanocytes |
| Tyrosinase Related Protein, TRP-1 | Melanocytes |
| Prostate Specific Antigen, PSA | Prostate Cancer |
| Albumin | Liver |
| Apolipoprotein | Liver |
| Plasminogen Activator | Liver |
| Inhibitor Type-1, PAI-1 | |
| Fatty Acid Binding | Colon Epithelial Cells |
| Insulin | Pancreatic Cells |
| Muscle Creatine Kinase, MCK | Muscle Cell |
| Myelin Basic Protein, MBP | Oligodendrocytes and Glial Cells |
| Glial Fibrillary Acidic | Glial Cells |
| Protein, GFAP | |
| Neural Specific Enolase | Nerve Cells |
| Immunoglobulin Heavy Chain | B-cells |
| Immunoglobulin Light Chain | B-cells, Activated T-cells |
| T-Cell Receptor | Lymphocytes |
| HLA DQα and DQβ | Lymphocytes |
| β-Interferon | Leukocytes; Lymphocytes Fibroblasts |
| Interlukin-2 | Activated T-cells |
| Platelet Derived Growth Factor | Erythrocytes |
| E2F-1 | Proliferating Cells |
| Cyclin A | Proliferating Cells |
| α-, β-Actin | Muscle Cells |
| Haemoglobin | Erythroid Cells |
| Elastase I | Pancreatic Cells |
| Neural Cell Adhesion | Neural Cells |
| Molecule, NCAM | |

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, the polypeptide can be expressed in bacterial cells such as E coli, insect cells, yeast or mammalian cells (such as Chinese hamster ovary cells (CHO), COS cells, NIH3T3 cells, NOS cells or PERC.6 cells). Other suitable host cells are known to those skilled in the art.

Vector DNA can be introduced into prokaryotic or eukaryotic cells via conventional transformation, infection or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (“Molecular Cloning: A Laboratory Manual” 2nd ed, Cold Spring Harbor Laboratory, Cold Spring Harbor Laboratory Press, Cold Spring Harbor, N.Y., 1989), and other laboratory manuals.

A host cell of the invention, such as a prokaryotic or eukaryotic host cell in culture, can be used to produce (i.e., express) IGF, IGFBP or ALS polypeptides. Accordingly, the invention further provides methods for producing polypeptides using the host cells of the invention. In one embodiment, the method comprises culturing the host cell of invention (into which a recombinant expression vector encoding a polypeptide has been introduced) in a suitable medium until the polypeptide is produced. In another embodiment, the method further comprises isolating the polypeptide from the medium or the host cell.

An enhancer-promoter used in a vector construct of the present invention can be any enhancer-promoter that drives expression in a cell to be transfected. By employing an enhancer-promoter with well-known properties, the level and pattern of gene product expression can be optimized.

A DNA molecule, gene or polynucleotide of the present invention can be incorporated into a vector by a number of techniques which are well known in the art. For instance, the vector pUC18 has been demonstrated to be of particular value Likewise, the related vectors M13mp18 and M13mp19 can be used in certain embodiments of the invention, in particular, in performing dideoxy sequencing.

An expression vector of the present invention is useful both as a means for preparing quantities of the polypeptide-encoding DNA itself, and as a means for preparing the encoded polypeptide and peptides. It is contemplated that where polypeptides of the invention are made by recombinant means, one can employ either prokaryotic or eukaryotic expression vectors as shuttle systems. However, prokaryotic systems are usually incapable of correctly processing precursor polypeptides and, in particular, such systems are incapable of correctly processing membrane associated eukaryotic polypeptides, and since eukaryotic polypeptides are anticipated using the teaching of the disclosed invention, one likely expresses such sequences in eukaryotic hosts. However, even where the DNA segment encodes a eukaryotic polypeptide, it is contemplated that prokaryotic expression can have some additional applicability. Therefore, the invention can be used in combination with vectors which can shuttle between the eukaryotic and prokaryotic cells. Such a system is described herein which allows the use of bacterial host cells as well as eukaryotic host cells.

Where expression of recombinant polypeptides is desired and a eukaryotic host is contemplated, it is most desirable to employ a vector such as a plasmid, that incorporates a eukaryotic origin of replication. Additionally, for the purposes of expression in eukaryotic systems, one desires to position the encoding sequence adjacent to and under the control of an effective eukaryotic promoter such as promoters used in combination with Chinese hamster ovary cells. To bring a coding sequence under control of a promoter, whether it is eukaryotic or prokaryotic, what is generally needed is to position the 5′ end of the translation initiation side of the proper translational reading frame of the polypeptide between about 1 and about 50 nucleotides 3′ of, or downstream, of the promoter chosen. Furthermore, where eukaryotic expression is anticipated, one would typically desire to incorporate into the transcriptional unit, which includes the polypeptide, an appropriate polyadenylation site.

The pCMV plasmids are a series of mammalian expression vectors of particular utility in the present invention. The vectors are designed for use in essentially all cultured cells and work extremely well in SV40-transformed simian COS cell lines. The pCMV1, 2, 3, and 5 vectors differ from each other in certain unique restriction sites in the polylinker region of each plasmid. The pCMV4 vector differs from these four plasmids in containing a translation enhancer in the sequence prior to the polylinker. While they are not directly derived from the pCMV1-5 series of vectors, the functionally similar pCMV6b and pCMV6c vectors are available from the Chiron Corp. (Emeryville, Calif.) and are identical except for the orientation of the polylinker region which is reversed in one relative to the other.

The universal components of the pCMV plasmids are as follows. The vector backbone is pTZ18R (Pharmacia), and contains a bacteriophage f1 origin of replication for production of single stranded DNA and an ampicillin-resistant gene. The CMV region consists of nucleotides −760 to +3 of the powerful promoter-regulatory region of the human cytomegalovirus (Towne stain) major immediate early gene (Thomsen et al., 1984; Boshart et al., 1985). The human growth hormone fragment (hGH) contains transcription termination and poly-adenylation signals representing sequences 1533 to 2157 of this gene (Seeburg, 1982). There is an Alu middle repetitive DNA sequence in this fragment. Finally, the SV40 origin of replication and early region promoter-enhancer derived from the pcD-X plasmid (HindIII to PstI fragment) described in (Okayama et al., 1983). The promoter in this fragment is oriented such that transcription proceeds away from the CMV/hGH expression cassette.

The pCMV plasmids are distinguishable from each other by differences in the polylinker region and by the presence or absence of the translation enhancer. The starting pCMV1 plasmid has been progressively modified to render an increasing number of unique restriction sites in the polylinker region. To create pCMV2, one of two EcoRI sites in pCMV1 were destroyed. To create pCMV3, pCMV1 was modified by deleting a short segment from the SV40 region (StuI to EcoRI), and in so doing made unique the PstI, SalI, and BamHI sites in the polylinker. To create pCMV4, a synthetic fragment of DNA corresponding to the 5′-untranslated region of an mRNA transcribed from the CMV promoter was added. The sequence acts as a translational enhancer by decreasing the requirements for initiation factors in polypeptide synthesis (Jobling et al., 1987; Browning et al., 1988). To create pCMV5, a segment of DNA (HpaI to EcoRI) was deleted from the SV40 origin region of pCMV1 to render unique all sites in the starting polylinker.

The pCMV vectors have been successfully expressed in simian COS cells, mouse L cells, CHO cells, and HeLa cells. In several side by side comparisons they have yielded 5- to 10-fold higher expression levels in COS cells than SV40-based vectors. The pCMV vectors have been used to express the LDL receptor, nuclear factor 1, GS alpha polypeptide, polypeptide phosphatase, synaptophysin, synapsin, insulin receptor, influenza hemagglutinin, androgen receptor, sterol 26-hydroxylase, steroid 17- and 21-hydroxylase, cytochrome P-450 oxidoreductase, beta-adrenergic receptor, folate receptor, cholesterol side chain cleavage enzyme, and a host of other cDNAs. It should be noted that the SV40 promoter in these plasmids can be used to express other genes such as dominant selectable markers. Finally, there is an ATG sequence in the polylinker between the HindIII and PstI sites in pCMU that can cause spurious translation initiation. This codon should be avoided if possible in expression plasmids. A paper describing the construction and use of the parenteral pCMV1 and pCMV4 vectors has been published (Anderson et al., 1989b).

In yet another embodiment, the present invention provides recombinant host cells transformed, infected or transfected with polynucleotides that encode polypeptides. Means of transforming or transfecting cells with exogenous polynucleotide such as DNA molecules are well known in the art and include techniques such as calcium-phosphate- or DEAE-dextran-mediated transfection, protoplast fusion, electroporation, liposome mediated transfection, direct microinjection and adenovirus infection (Sambrook, Fritsch and Maniatis, 1989).

The most widely used method is transfection mediated by either calcium phosphate or DEAE-dextran. Although the mechanism remains obscure, it is believed that the transfected DNA enters the cytoplasm of the cell by endocytosis and is transported to the nucleus. Depending on the cell type, up to 90% of a population of cultured cells can be transfected at any one time. Because of its high efficiency, transfection mediated by calcium phosphate or DEAE-dextran is the method of choice for experiments that require transient expression of the foreign DNA in large numbers of cells. Calcium phosphate-mediated transfection is also used to establish cell lines that integrate copies of the foreign DNA, which are usually arranged in head-to-tail tandem arrays into the host cell genome.

In the protoplast fusion method, protoplasts derived from bacteria carrying high numbers of copies of a plasmid of interest are mixed directly with cultured mammalian cells. After fusion of the cell membranes (usually with polyethylene glycol), the contents of the bacteria are delivered into the cytoplasm of the mammalian cells and the plasmid DNA is transported to the nucleus. Protoplast fusion is not as efficient as transfection for many of the cell lines that are commonly used for transient expression assays, but it is useful for cell lines in which endocytosis of DNA occurs inefficiently. Protoplast fusion frequently yields multiple copies of the plasmid DNA tandemly integrated into the host chromosome.

The application of brief, high-voltage electric pulses to a variety of mammalian and plant cells leads to the formation of nanometer-sized pores in the plasma membrane. DNA is taken directly into the cell cytoplasm either through these pores or as a consequence of the redistribution of membrane components that accompanies closure of the pores. Electroporation can be extremely efficient and can be used both for transient expression of cloned genes and for establishment of cell lines that carry integrated copies of the gene of interest. Electroporation, in contrast to calcium phosphate-mediated transfection and protoplast fusion, frequently gives rise to cell lines that carry one, or at most a few, integrated copies of the foreign DNA.

Liposome transfection involves encapsulation of DNA and RNA within liposomes, followed by fusion of the liposomes with the cell membrane. The mechanism of how DNA is delivered into the cell is unclear but transfection efficiencies can be as high as 90%.

Direct microinjection of a DNA molecule into nuclei has the advantage of not exposing DNA to cellular compartments such as low-pH endosomes. Microinjection is therefore used primarily as a method to establish lines of cells that carry integrated copies of the DNA of interest.

The use of adenovirus as a vector for cell transfection is well known in the art. Adenovirus vector-mediated cell transfection has been reported for various cells (Stratford-Perricaudet, et al. 1992).

D. IGFBP and IGF Antibodies

In certain embodiments, the invention is directed to methods of screening for compounds which dissociate an IGF/IGFBP dimer or an IGF/IGFBP/ALS trimer. It is contemplated certain embodiments, that antibodies directed to either IGF or IGFBP will be particularly useful in such screening methods. Thus, the present invention provides antibodies immunoreactive with IGF or IGFBP polypeptides. Preferably, the antibodies of the invention are monoclonal antibodies. Means for preparing and characterizing antibodies are well known in the art (see, e.g., Antibodies “A Laboratory Manual, E. Howell and D. Lane, Cold Spring Harbor Laboratory, 1988).

Briefly, a polyclonal antibody is prepared by immunizing an animal with an immunogen comprising a polypeptide or polynucleotide of the present invention, and collecting antisera from that immunized animal. A wide range of animal species can be used for the production of antisera. Typically an animal used for production of anti-antisera is a rabbit, a mouse, a rat, a hamster or a guinea pig. Because of the relatively large blood volume of rabbits, a rabbit is a preferred choice for production of polyclonal antibodies.

As is well known in the art, a given polypeptide or polynucleotide may vary in its immunogenicity. It is often necessary therefore to couple the immunogen (e.g., a polypeptide or polynucleotide) of the present invention with a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers.

Means for conjugating a polypeptide or a polynucleotide to a carrier polypeptide are well known in the art and include glutaraldehyde, m-maleimidobencoyl-N-hydroxysuccinimide ester, carbodiimide and bis-biazotized benzidine.

As is also well known in the art, immunogencity to a particular immunogen can be enhanced by the use of non-specific stimulators of the immune response known as adjuvants. Exemplary and preferred adjuvants include complete Freund's adjuvant, incomplete Freund's adjuvants and aluminum hydroxide adjuvant.

The amount of immunogen used for the production of polyclonal antibodies varies inter alia, upon the nature of the immunogen as well as the animal used for immunization. A variety of routes can be used to administer the immunogen (subcutaneous, intramuscular, intradermal, intravenous and intraperitoneal). The production of polyclonal antibodies is monitored by sampling blood of the immunized animal at various points following immunization. When a desired level of immunogenicity is obtained, the immunized animal can be bled and the serum isolated and stored.

A monoclonal antibody of the present invention can be readily prepared through use of well-known techniques such as those exemplified in U.S. Pat. No. 4,196,265, herein incorporated by reference. Typically, a technique involves first immunizing a suitable animal with a selected antigen (e.g., a polypeptide or polynucleotide of the present invention) in a manner sufficient to provide an immune response. Rodents such as mice and rats are preferred animals. Spleen cells from the immunized animal are then fused with cells of an immortal myeloma cell. Where the immunized animal is a mouse, a preferred myeloma cell is a murine NS-1 myeloma cell.

The fused spleen/myeloma cells are cultured in a selective medium to select fused spleen/myeloma cells from the parental cells. Fused cells are separated from the mixture of non-fused parental cells, e.g., by the addition of agents that block the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides. Where azaserine is used, the media is supplemented with hypoxanthine.

This culturing provides a population of hybridomas from which specific hybridomas are selected. Typically, selection of hybridomas is performed by culturing the cells by single-clone dilution in microtiter plates, followed by testing the individual clonal supernatants for reactivity with an antigen-polypeptide. The selected clones can then be propagated indefinitely to provide the monoclonal antibody.

By way of specific example, to produce an antibody of the present invention, mice are injected intraperitoneally with between about 1-200 μg of an antigen comprising a polypeptide of the present invention. B lymphocyte cells are stimulated to grow by injecting the antigen in association with an adjuvant such as complete Freund's adjuvant (a non-specific stimulator of the immune response containing killed Mycobacterium tuberculosis). At some time (e.g., at least two weeks) after the first injection, mice are boosted by injection with a second dose of the antigen mixed with incomplete Freund's adjuvant.

A few weeks after the second injection, mice are tail bled and the sera titered by immunoprecipitation against radiolabeled antigen. Preferably, the process of boosting and titering is repeated until a suitable titer is achieved. The spleen of the mouse with the highest titer is removed and the spleen lymphocytes are obtained by homogenizing the spleen with a syringe. Typically, a spleen from an immunized mouse contains approximately 5×10⁷ to 2×10⁸ lymphocytes.

Mutant lymphocyte cells known as myeloma cells are obtained from laboratory animals in which such cells have been induced to grow by a variety of well-known methods. Myeloma cells lack the salvage pathway of nucleotide biosynthesis. Because myeloma cells are tumor cells, they can be propagated indefinitely in tissue culture, and are thus denominated immortal. Numerous cultured cell lines of myeloma cells from mice and rats, such as murine NS-1 myeloma cells, have been established.

Myeloma cells are combined under conditions appropriate to foster fusion with the normal antibody-producing cells from the spleen of the mouse or rat injected with the antigen/polypeptide of the present invention. Fusion conditions include, for example, the presence of polyethylene glycol. The resulting fused cells are hybridoma cells. Like myeloma cells, hybridoma cells grow indefinitely in culture.

Hybridoma cells are separated from unfused myeloma cells by culturing in a selection medium such as HAT media (hypoxanthine, aminopterin, thymidine). Unfused myeloma cells lack the enzymes necessary to synthesize nucleotides from the salvage pathway because they are killed in the presence of aminopterin, methotrexate, or azaserine. Unfused lymphocytes also do not continue to grow in tissue culture. Thus, only cells that have successfully fused (hybridoma cells) can grow in the selection media.

Each of the surviving hybridoma cells produces a single antibody. These cells are then screened for the production of the specific antibody immunoreactive with an antigen/polypeptide of the present invention. Single cell hybridomas are isolated by limiting dilutions of the hybridomas. The hybridomas are serially diluted many times and, after the dilutions are allowed to grow, the supernatant is tested for the presence of the monoclonal antibody. The clones producing that antibody are then cultured in large amounts to produce an antibody of the present invention in convenient quantity.

By use of a monoclonal antibody of the present invention, specific polypeptides of the invention can be recognized as antigens, and thus identified. Once identified, those polypeptides can be isolated and purified by techniques such as antibody-affinity chromatography. In antibody-affinity chromatography, a monoclonal antibody is bound to a solid substrate and exposed to a solution containing the desired antigen. The antigen is removed from the solution through an immunospecific reaction with the bound antibody. The polypeptide is then easily removed from the substrate and purified.

Additionally, examples of methods and reagents particularly amenable for use in generating and screening an antibody display library can be found in, for example, U.S. Pat. No. 5,223,409; International Application No. WO 92/18619; International Application No. WO 91/17271; International Application No. WO 92/20791; International Application No. WO 92/15679; International Application No. WO 93/01288; International Application No. WO 92/01047; International Application No. WO 92/09690; International Application No. WO 90/02809.

Additionally, recombinant anti-IGF or -IGFBP antibodies, such as chimeric and humanized monoclonal antibodies, comprising both human and non-human fragments, which can be made using standard recombinant DNA techniques, are within the scope of the invention. Such chimeric and humanized monoclonal antibodies can be produced by recombinant DNA techniques known in the art, for example using methods described in U.S. Pat. No. 6,054,297; European Application Nos. EP 184,187; EP 171,496; EP 173,494; International Application No. WO 86/01533; U.S. Pat. No. 4,816,567; and European Application No. EP 125,023.

An antibody (e.g., monoclonal antibody) can be used to isolate the polypeptides (e.g., IGF or IGFBP) by standard techniques, such as affinity chromatography or immunoprecipitation. An anti-IGF antibody for example, can facilitate the purification of recombinantly produced IGF polypeptide expressed in host cells. Moreover, an anti-IGF or anti-IGFBP antibody can be used to detect IGF or IGFBP polypeptide (e.g., in a cellular lysate or cell supernatant) in order to evaluate the abundance of the polypeptide, evaluate binding properties of the polypeptide or the pattern of expression of the polypeptide.

Anti-IGF or -IGFBP antibodies can be used diagnostically to monitor protein levels, e.g., to determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, P-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and acquorin, and examples of suitable radioactive material include ¹²⁵I, ¹³¹I, ¹⁵S or ³H.

E. Transgenic Animals

In certain embodiments, the invention pertains to nonhuman animals with somatic and germ cells having a functional disruption of at least one, and more preferably both, alleles of an endogenous IGF and/or IGFBP and/or ALS gene of the present invention. Accordingly, the invention provides viable animals having a mutated IGF and/or IGFBP and/or ALS gene, and thus lacking IGF and/or IGFBP and/or ALS activity. These animals will produce substantially reduced amounts of a IGF and/or IGFBP and/or ALS in response to stimuli that produce normal amounts of a IGF and/or IGFBP and/or ALS in wild type control animals. The animals of the invention are useful, for example, as standard controls by which to evaluate IGF and/or IGFBP and/or ALS modulatory compounds, as recipients of a normal human IGF and/or IGFBP and/or ALS gene to thereby create a model system for screening human IGF and/or IGFBP and/or ALS modulators in vivo, and to identify disease states for treatment with IGF and/or IGFBP and/or ALS modulators. The animals are also useful as controls for studying the effect of modulators on IGF and/or IGFBP and/or ALS.

In the transgenic nonhuman animal of the invention, the IGF and/or IGFBP and/or ALS gene preferably is disrupted by homologous recombination between the endogenous allele and a mutant IGF and/or IGFBP and/or ALS polynucleotide, or portion thereof, that has been introduced into an embryonic stem cell precursor of the animal. The embryonic stem cell precursor is then allowed to develop, resulting in an animal having a functionally disrupted IGF and/or IGFBP and/or ALS gene. As used herein, a “transgenic animal” is a non-human animal, preferably a mammal, more preferably a rodent such as a rat or mouse, in which one or more of the cells of the animal include a transgene. Other examples of transgenic animals include non-human primates, sheep, dogs, cows, goats, chickens, amphibians, and the like. The animal may have one IGF and/or IGFBP and/or ALS gene allele functionally disrupted (i.e., the animal may be heterozygous for the mutation), or more preferably, the animal has both IGF and/or IGFBP and/or ALS gene alleles functionally disrupted (i.e., the animal can be homozygous for the mutation).

In one embodiment of the invention, functional disruption of both IGF and/or IGFBP and/or ALS gene alleles produces animals in which expression of the IGF and/or IGFBP and/or ALS gene product in cells of the animal is substantially absent relative to non-mutant animals. In another embodiment, the IGF and/or IGFBP and/or ALS gene alleles can be disrupted such that an altered (i.e., mutant) IGF and/or IGFBP and/or ALS gene product is produced in cells of the animal. A preferred nonhuman animal of the invention having a functionally disrupted IGF and/or IGFBP and/or ALS gene is a mouse. Given the essentially complete inactivation of IGF and/or IGFBP and/or ALS function in the homozygous animals of the invention and the about 50% inhibition of IGF and/or IGFBP and/or ALS function in the heterozygous animals of the invention, these animals are useful as positive controls against which to evaluate the effectiveness of IGF and/or IGFBP and/or ALS modulators.

Additionally, the animals of the invention are useful for determining whether a particular disease condition involves the action of IGF and/or IGFBP and/or ALS and thus can be treated by an IGF and/or IGFBP and/or ALS modulator. For example, an attempt can be made to induce a disease condition in an animal of the invention having a functionally disrupted IGF and/or IGFBP and/or ALS gene. Subsequently, the susceptibility or resistance of the animal to the disease condition can be determined. A disease condition that is treatable with an IGF and/or IGFBP and/or ALS modulatory compound can be identified based upon resistance of an animal of the invention to the disease condition.

Another aspect of the invention pertains to a transgenic nonhuman animal having a functionally disrupted endogenous IGF and/or IGFBP and/or ALS gene, but which also carries in its genome, and expresses, a transgene encoding a heterologous IGF and/or IGFBP and/or ALS (i.e., a IGF and/or IGFBP and/or ALS from another species). Preferably, the animal is a mouse and the heterologous IGF and/or IGFBP and/or ALS is a human IGF and/or IGFBP and/or ALS. An animal of the invention which has been reconstituted with human IGF and/or IGFBP and/or ALS can be used to identify agents that dissociate human IGF and/or IGFBP and/or ALS in vivo. For example, a stimulus that induces production and/or activity of IGF and/or IGFBP and/or ALS can be administered to the animal in the presence and absence of an agent to be tested and the IGF and/or IGFBP and/or ALS response in the animal can be measured.

As used herein, a “transgene” is exogenous DNA which is integrated into the genome of a cell from which a transgenic animal develops and which remains in the genome of the mature animal, thereby directing the expression of an encoded gene product in one or more cell types or tissues of the transgenic animal.

Yet another aspect of the invention pertains to a polynucleotide construct for functionally disrupting a IGF or IGFBP or ALS gene in a host cell. The nucleic acid construct comprises: a) a nonhomologous replacement portion; b) a first homology region located upstream of the nonhomologous replacement portion, the first homology region having a nucleotide sequence with substantial identity to a first IGF or IGFBP or ALS gene sequence; and c) a second homology region located downstream of the nonhomologous replacement portion, the second homology region having a nucleotide sequence with substantial identity to a second IGF or IGFBP or ALS gene sequence, the second IGF or IGFBP or ALS gene sequence having a location downstream of the first IGF or IGFBP or ALS gene sequence in a naturally occurring endogenous IGF or IGFBP or ALS gene. Additionally, the first and second homology regions are of sufficient length for homologous recombination between the nucleic acid construct and an endogenous IGF or IGFBP or ALS gene in a host cell when the nucleic acid molecule is introduced into the host cell. As used herein, a “homologous recombinant animal” is a non-human animal, preferably a mammal, more preferably a mouse, in which an endogenous IGF or IGFBP or ALS gene has been altered by homologous recombination between the endogenous gene and an exogenous DNA molecule introduced into a cell of the animal, e.g., an embryonic cell of the animal, prior to development of the animal.

In a preferred embodiment, the nonhomologous replacement portion comprises a positive selection expression cassette, preferably including a neomycin phosphotransferase gene operatively linked to a regulatory element(s). In another preferred embodiment, the nucleic acid construct also includes a negative selection expression cassette distal to either the upstream or downstream homology regions. A preferred negative selection cassette includes a herpes simplex virus thymidine kinase gene operatively linked to a regulatory element(s). Another aspect of the invention pertains to recombinant vectors into which the nucleic acid construct of the invention has been incorporated.

Yet another aspect of the invention pertains to host cells into which the nucleic acid construct of the invention has been introduced to thereby allow homologous recombination between the nucleic acid construct and an endogenous IGF or IGFBP or ALS gene of the host cell, resulting in functional disruption of the endogenous IGF or IGFBP or ALS gene. The host cell can be a mammalian cell that normally expresses IGF or IGFBP or ALS, such as a human neuron, or a pluripotent cell, such as a mouse embryonic stem cell. Further development of an embryonic stem cell into which the nucleic acid construct has been introduced and homologously recombined with the endogenous IGF or IGFBP or ALS gene produces a transgenic nonhuman animal having cells that are descendant from the embryonic stem cell and thus carry the IGF or IGFBP or ALS gene disruption in their genome. Animals that carry the IGF or IGFBP or ALS gene disruption in their germline can then be selected and bred to produce animals having the IGF or IGFBP or ALS gene disruption in all somatic and germ cells. Such mice can then be bred to homozygosity for the IGF or IGFBP or ALS gene disruption.

It is contemplated that in some instances the genome of a transgenic animal of the present invention will have been altered through the stable introduction of one or more of the IGF or IGFBP or ALS polynucleotide compositions described herein, either native, synthetically modified or mutated. As described herein, a “transgenic animal” refers to any animal, preferably a non-human mammal (e.g. mouse, rat, rabbit, squirrel, hamster, rabbits, guinea pigs, pigs, micro-pigs, prairie, baboons, squirrel monkeys and chimpanzees, etc), bird or an amphibian, in which one or more cells contain heterologous nucleic acid introduced by way of human intervention, such as by transgenic techniques well known in the art. The nucleic acid is introduced into the cell, directly or indirectly, by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA.

The host cells of the invention can also be used to produce non-human transgenic animals. The non-human transgenic animals can be used in screening assays designed to identify agents or compounds, e.g., drugs, pharmaceuticals, etc., which are capable of ameliorating detrimental symptoms of selected disorders such as nervous system disorders, e.g., psychiatric disorders or disorders affecting circadian rhythms and the sleep-wake cycle. For example, in one embodiment, a host cell of the invention is a fertilized oocyte or an embryonic stem cell into which IGF or IGFBP or ALS polypeptide-coding sequences have been introduced. Such host cells can then be used to create non-human transgenic animals in which exogenous IGF or IGFBP or ALS gene sequences have been introduced into their genome or homologous recombinant animals in which endogenous IGF or IGFBP or ALS gene sequences have been altered. Such animals are useful for studying the function and/or activity of a IGF or IGFBP or ALS polypeptide and for identifying and/or evaluating modulators of IGF or IGFBP or ALS polypeptide activity.

A transgenic animal of the invention can be created by introducing IGF or IGFBP or ALS polypeptide encoding nucleic acid into the male pronuclei of a fertilized oocyte, e.g., by microinjection, retroviral infection, and allowing the oocyte to develop in a pseudopregnant female foster animal. The human IGF or IGFBP or ALS cDNA sequence can be introduced as a transgene into the genome of a non-human animal.

Moreover, a non-human homologue of the human IGF or IGFBP or ALS gene, such as a mouse IGF or IGFBP or ALS gene, can be isolated based on hybridization to the human IGF or IGFBP or ALS cDNA (described above) and used as a transgene. Intronic sequences and polyadenylation signals can also be included in the transgene to increase the efficiency of expression of the transgene. A tissue-specific regulatory sequence(s) can be operably linked to the IGF or IGFBP or ALS transgene to direct expression of a IGF or IGFBP or ALS polypeptide to particular cells. Methods for generating transgenic animals via embryo manipulation and microinjection, particularly animals such as mice, have become conventional in the art and are described, for example, in U.S. Pat. No. 4,736,866, U.S. Pat. No. 4,870,009, U.S. Pat. No. 4,873,191 and in Hogan, 1986. Similar methods are used for production of other transgenic animals. A transgenic founder animal can be identified based upon the presence of the IGF or IGFBP or ALS transgene in its genome and/or expression of IGF or IGFBP or ALS mRNA in tissues or cells of the animals. A transgenic founder animal can then be used to breed additional animals carrying the transgene. Moreover, transgenic animals carrying a transgene encoding a IGF or IGFBP or ALS polypeptide can further be bred to other transgenic animals carrying other transgenes.

To create a homologous recombinant animal, a vector is prepared which contains at least a fragment of a IGF or IGFBP or ALS gene into which a deletion, addition or substitution has been introduced to thereby alter, e.g., functionally disrupt, the IGF or IGFBP or ALS gene. The IGF or IGFBP or ALS gene can be a human gene (e.g., from a human genomic clone isolated from a human genomic library screened with the cDNA of SEQ ID NO:1, SEQ ID NO:3, SEQ ID NO:5, SEQ ID NO:7 or SEQ ID NO:9), but more preferably is a non-human homologue of a human IGF or IGFBP or ALS gene. For example, a mouse IGF or IGFBP or ALS gene can be isolated from a mouse genomic DNA library using the IGF or IGFBP or ALS cDNA as a probe. The mouse IGF or IGFBP or ALS gene then can be used to construct a homologous recombination vector suitable for altering an endogenous IGF or IGFBP or ALS gene in the mouse genome. In a preferred embodiment, the vector is designed such that, upon homologous recombination, the endogenous IGF or IGFBP or ALS gene is functionally disrupted (i.e., no longer encodes a functional protein; also referred to as a “knock out” vector.

Alternatively, the vector can be designed such that, upon homologous recombination, the endogenous IGF or IGFBP or ALS gene is mutated or otherwise altered but still encodes functional protein (e.g., the upstream regulatory region can be altered to thereby alter the expression of the endogenous IGF or IGFBP or ALS polypeptide). In the homologous recombination vector, the altered fragment of the IGF or IGFBP or ALS gene is flanked at its 5′ and 3′ ends by additional nucleic acid of the IGF or IGFBP or ALS to allow for homologous recombination to occur between the exogenous IGF or IGFBP or ALS gene carried by the vector and an endogenous IGF or IGFBP or ALS gene in an embryonic stem cell. The additional flanking IGF or IGFBP or ALS nucleic acid is of sufficient length for successful homologous recombination with the endogenous gene.

Typically, several kilobases of flanking DNA (both at the 5′ and 3′ ends) are included in the vector (see e.g., Thomas and Capecchi, 1987, for a description of homologous recombination vectors). The vector is introduced into an embryonic stem cell line (e.g., by electroporation) and cells in which the introduced IGF or IGFBP or ALS gene has homologously recombined with the endogenous IGF or IGFBP or ALS gene are selected (see e.g., Li et al., 1992). The selected cells are then injected into a blastocyst of an animal (e.g., a mouse) to form aggregation chimeras (see e.g., Bradley, 1987, pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, 1991; and in PCT International Publication Nos. WO 90/11354; WO 91/01140; and WO 93/04169.

In another embodiment, transgenic non-human animals can be produced which contain selected systems which allow for regulated expression of the transgene. One example of such a system is the cre/loxP recombinase system of bacteriophage PL. For a description of the cre/loxP recombinase system, see, e.g., Lakso et al., 1992. Another example of a recombinase system is the FLP recombinase system of Saccharomyces cerevisiae (O'Gonnan et al., 1991). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al., 1997, and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring borne of this female foster animal will be a clone of the animal from which the cell, e.g., the somatic cell, is isolated.

F. Uses and Methods of the Invention

The polypeptides, polypeptide fragments, peptide mimetics, small molecules, antisense molecules, antibodies and the like, can be used in one or more of the following methods: a) drug screening assays; b) diagnostic assays, particularly in disease identification; c) methods of treatment; and d) monitoring of effects during clinical trials. A polypeptide of the invention (e.g., IGFBP-2) can be used as a drug target for developing agents (e.g., small molecules, peptides) to dissociate IGF/IGFBP polypeptide interactions. Similarly an antisense RNA molecule can be used to modulate IGFBP expression, thereby reducing IGFBP polypeptide levels. Moreover, the anti-IGF or anti-IGFBP antibodies of the invention can be used to detect and isolate polypeptides, polypeptide fragments and to modulate IGFBP polypeptide activity.

1. Drug Screening Assays

The invention provides methods for identifying compounds or agents that can be used to treat neurological disorders by dissociating IGF/IGFBP and/or IGF/IGFBP/ALS complexes. These methods are also referred to herein as drug screening assays and typically include the step of screening a candidate/test compound or agent to identify compounds that dissociate or prevent IGF-IGFBP non-covalent binding or association. Candidate/test compounds or agents which dissociate or prevent IGF-IGFBP non-covalent binding interactions can be used as “drugs” to treat neurological disorders associated with low concentrations of IGF polypeptides, particularly in the brain. Candidate/test compounds include, for example, 1) peptides such as soluble peptides, including Ig-tailed fusion peptides and members of random peptide libraries and combinatorial chemistry-derived molecular libraries made of D- and/or L-configuration amino acids; 2) phosphopeptides (e.g., members of random and partially degenerate directed phosphopeptide libraries, see, e.g., Songyang et al., 1993; 3) antibodies (e.g., polyclonal, monoclonal, humanized, anti-idiotypic, chimeric, and single chain antibodies, as well as Fab, F(ab′)2, Fab expression library fragments, and epitope-binding fragments of antibodies; and 4) small molecules, organic and inorganic (e.g., molecules obtained from combinatorial and natural product libraries).

In one embodiment, the invention provides assays for screening candidate/test compounds which interact with (e.g., bind to) an IGF or IGFBP polypeptide. Typically, the assays are recombinant cell based or cell-free assays which include the steps of combining a cell expressing an IGF and IGFBP polypeptide or a bioactive fragment thereof, or combining IGF and IGFBP polypeptides, adding a candidate/test compound, e.g., under conditions which allow for interaction of (e.g., binding of) the candidate/test compound to the IGF or IGFBP polypeptide to form a complex, and detecting the ability of the candidate compound to dissociate the IGF/IGFBP complex (e.g., see Examples 7, 9 and 10).

Detection of IGF/IGFBP complex dissociation can include direct quantitation of the complex using methods such as those described in Example 7. A statistically significant change, such as a decrease in the interaction of the IGF polypeptide and IGFBP in the presence of a candidate compound (relative to what is detected in the absence of the candidate compound), is indicative of a modulation of the interaction between the IGF and IGFBP polypeptides. Modulation of the formation of complexes can be quantitated using, for example, an immunoassay.

2. Diagnostic Assays

The invention further provides a method for identifying an individual susceptible to a neurological disorder by detecting the presence of an IGFBP nucleic acid molecule, or fragment thereof, in a biological sample, as described below. The method involves contacting the biological sample with a compound or an agent capable of detecting mRNA such that the presence of an IGFBP encoding nucleic acid molecule is detected in the biological sample. A preferred agent for detecting IGFBP mRNA is a labeled or labelable nucleic acid probe capable of hybridizing to IGFBP mRNA. The nucleic acid probe can be, for example, the full-length cDNA, or a fragment thereof, such as an oligonucleotide of at least 15, 30, 50, 100, 250 or 500 nucleotides in length and sufficient to specifically hybridize under stringent conditions to IGFBP mRNA.

The term “biological sample” is intended to include tissues, cells and biological fluids isolated from a subject, as well as tissues, cells and fluids present within a subject. That is, the detection method of the invention can be used to detect IGFBP mRNA or protein in a biological sample in vitro as well as in vivo. For example, in vitro techniques for detection of IGFBP mRNA include Northern hybridizations and in situ hybridizations. In vitro techniques for detection of IGF or IGFBP polypeptide include enzyme linked immunosorbent assays (ELISAs), Western blots, immunoprecipitations and immunofluorescence. In vivo techniques for detection may include imaging techniques such as magnetic resonance imaging (MR)I or positron emission tomography (PET) scan.

3. Neurological Disorders

Another aspect of the invention pertains to methods for treating a subject, (e.g., a human) having a neurological disorder characterized by (or associated with) reduced IGF polypeptide concentrations (i.e., reduced concentrations of unbound, active IGF), particularly reduced concentrations in the CNS. These methods include the step of administering a small molecule, a peptide, an antibody or an antisense RNA molecule, which modulates the concentration of free IGF (i.e., unbound, active IGF). The terms “treating” or “treatment,” as used herein, refer to reduction or alleviation of at least one adverse effect or symptom of a disorder or disease, e.g., a disorder or disease characterized by, or associated with, reduced IGF polypeptide concentrations.

Thus, in particular embodiments, the invention is directed to methods and compositions for the treatment of various neurological diseases or disorders including, but not limited to, neuropsychiatric disorders such as schizophrenia, delirium, bipolar, depression, anxiety, panic disorders; urinary retention; ulcers; allergies; benign prostatic hypertrophy; and dyskinesias, such as Huntington's disease or Gilles dela Tourett's syndrome

In certain embodiments, the invention is directed to methods and compositions for treating disorders involving the brain, including, but not limited to, disorders involving neurons, disorders involving glia, such as astrocytes, oligodendrocytes, ependymal cells, and microglia; cerebral edema, raised intracranial pressure and herniationk, and hydrocephalus; malformations and developmental diseases, such as neural tube defects, forebrain anomalies, posterior fossak anomalies, and syringomyelia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-borne (Arbo) viral encephalitis, Herpes simplex virus Type 1, Herpes simplex virus Type 2, Varicella-zoster virus (Herpes zoster), cytomegalovirus, poliomyelitis, rabies, and human immunodeficiency virus 1, including FHV-1 meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer disease and Pick disease, degenerative diseases of basal ganglia and brain stem, including Parkinsonism, idiopathic Parkinson disease (paralysis agitans), progressive supranuclear palsy, corticobasal degeneration, multiple system atrophy, including striatonigral degeneration, Shy-Drager syndrome, and olivopontocerebellar atrophy, and Huntington disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophyk, adrenoleukodystrophy, Elizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin BI) deficiency and vitamin B12 deficiency, neurologic sequelae of metabolic disturbances, including hypoglycernia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastoma multiforme, pilocytic astrocytoma, pleomorphic xanthoastrocytorna, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), neurocutaneous syndromes (phakomatoses), including neurofibromotosis, including Type I neurofibromatosis (NFI) and TYPE 2 neurofibromatosis (NF2), tuberous sclerosis, and Von Hippel-Lindau disease, and neuropsychiatric disorders, such as schizophrenia, bipolar, depression, anxiety and panic disorders.

4. Pharmaceutical Compositions

The nucleic acids, polypeptides, polypeptide fragments, small anti-IGFBP antibodies and the like (referred to herein as “active compounds”) of the invention can be incorporated into pharmaceutical compositions suitable for administration to a subject, e.g., a human. Such compositions typically comprise the nucleic acid molecule, protein, modulator, or antibody and a pharmaceutically acceptable carrier. As used herein, the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressured container or dispenser which contains a suitable propellant, e.g., a gas such as carbon dioxide, or a nebulizer. Systemic administration can also be by transmucosal or transdermal means. For transmucosal or transdermal administration, penetrants appropriate for the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams, as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems.

Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811 which is incorporated by reference herein in its entirety.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by, and directly dependent, on the unique characteristics of the active compound and the particular therapeutic effect to be achieved and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al., 1994). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system. The pharmaceutical compositions can be included in a container, pack, or dispenser together with instructions for administration.

All patents and publications cited herein are incorporated by reference.

### EXAMPLES

The following examples are carried out using standard techniques, which are well known and routine to those of skill in the art, except where otherwise described in detail. The following examples are presented for illustrative purpose, and should not be construed in any way limiting the scope of this invention.

### Example 1

### IGFBP-5 mRNA Shows Unique Expression in the Mouse Dentate Gyrus

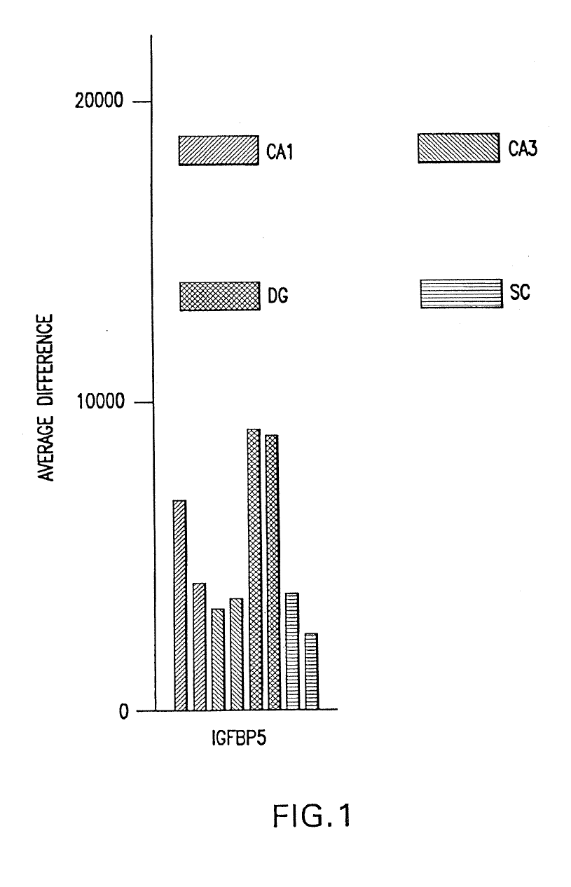

The dentate gyrus is one of the unique areas in the brain that demonstrates neurogenesis. Analysis of microarray data, which compared mouse dentate gyrus (DG) with CA1, CA3 and spinal cord, demonstrated IGFBP-5 enriched expression in DG compared to other regions by microarray (FIG. 1A). This finding was observed in independent groups of mice and was confirmed by both Taqman real-time PCR (data not shown) and in situ hybridization (data not shown). In some model systems, IGFBP-5 potentiates the effect of IGF-I (Duan and Clemmons, 1998) although this has not been determined in the CNS. This data supports the idea that the relationship between IGFBP-5 and IGF-I may be directly important in neurogenesis. Also intriguing is the observation that IGF-I regulates IGFBP-5 gene expression in the brain (Ye and D'Ercole, 1998), so the enhanced IGFBP-5 in the dentate gyrus may be secondary to increased IGF-I activity in this region.

### Example 2

### IGFBP-2 mRNA Shows Increased Expression in Fibroblasts from Subjects with Major Depression

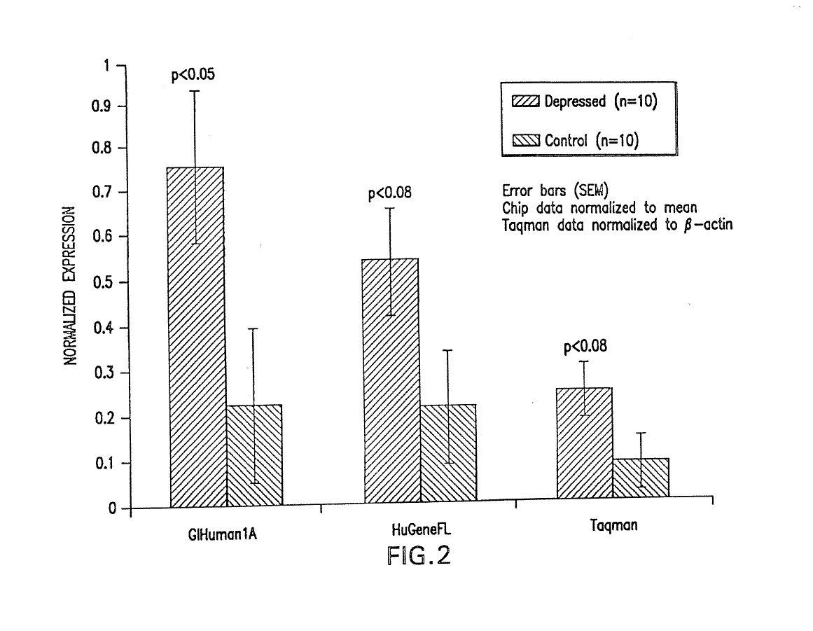

Psychiatric disease has effects on gene expression in peripheral tissues (Lesch et al., 1996). It has been has observed that fibroblast cell lines derived from skin biopsies from subjects with major depression show biochemical differences in signal transduction pathways when compared with cells from control subjects (Fridolin Sulser, unpublished data). To identify transcriptional differences between these two populations, the cell lines were profiled by microarray. IGFBP-2 showed a statistically significant increase in expression in the depressed population. This finding was reproduced using two microarray designs, which have different probe sequences and was confirmed by Taqman real-time PCR (FIG. 2). These data indicate that IGFBP-2 mRNA or protein levels in the periphery (from serum, leukocytes or skin biopsy) may be used as a diagnostic marker to diagnose depression in human subjects.

### Example 3

### IGFBP-2 mRNA Shows Slightly Increased Expression in Brain Tissue from Subjects with Major Depression

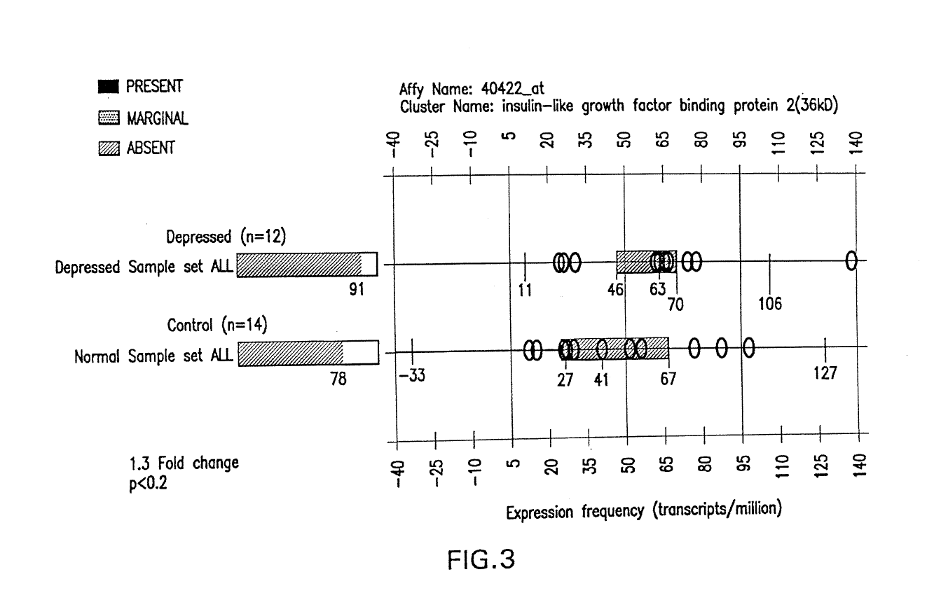

Human brain tissues of Brodmann area 21 were obtained from the Stanley Foundation and profiled by microarray (FIG. 3). A slight increase in IGFBP-2 was noted. Although this did not approach statistical significance (p<0.2), the trend was the same as that seen in the fibroblasts for this gene.

### Example 4

### IGF-I mRNA Shows Increased Expression in C6 Glioma Cell Lines

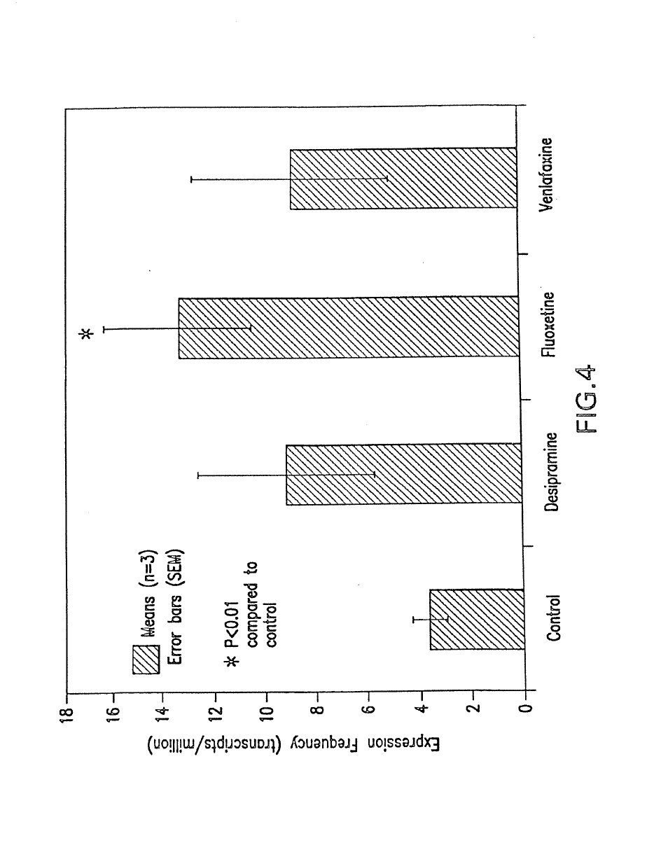

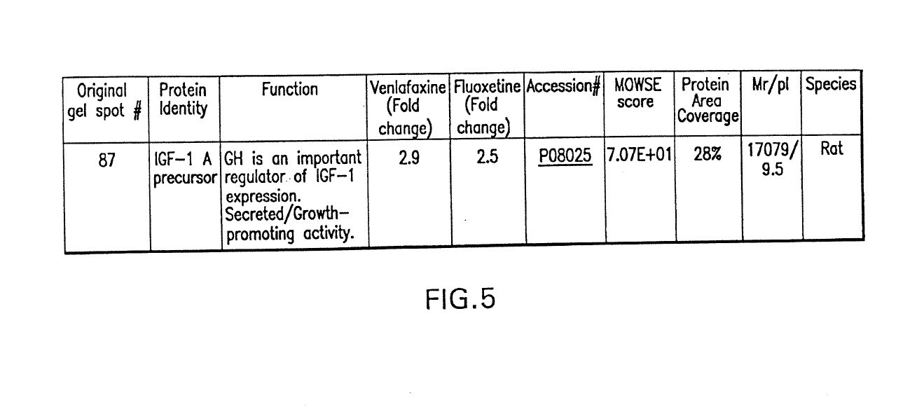

Quiescent C6 glioma cell lines were treated with fluoxetine, desipramine or venlafaxine for 24 hours and profiled by microarray; each demonstrated increased expression of IGF-I mRNA (FIG. 4). Glial cell cultures were used because they lack endogenous serotonin and norepinephrine transporter mechanisms. Thus, any transcriptional effects caused by the antidepressant drugs are due to actions beyond the level of the serotonin and norepinephrine receptors and respective transporters. Antidepressant drugs have been shown to enhance neurogenesis (Malberg et al 2000) and peripheral infusion of IGF-I selectively induces neurogenesis in the dentate gyrus (Aberg et al., 2000). Antidepressant drugs may therefore be acting on the glia, which produces the neurotrophic factor IGF-I, which acts in a juxtacrine manner to stimulate neurogenesis.

### Example 5

### IGF-IA Protein Shows Increased Expression in Antidepressant-Treated Rat Hippocampus

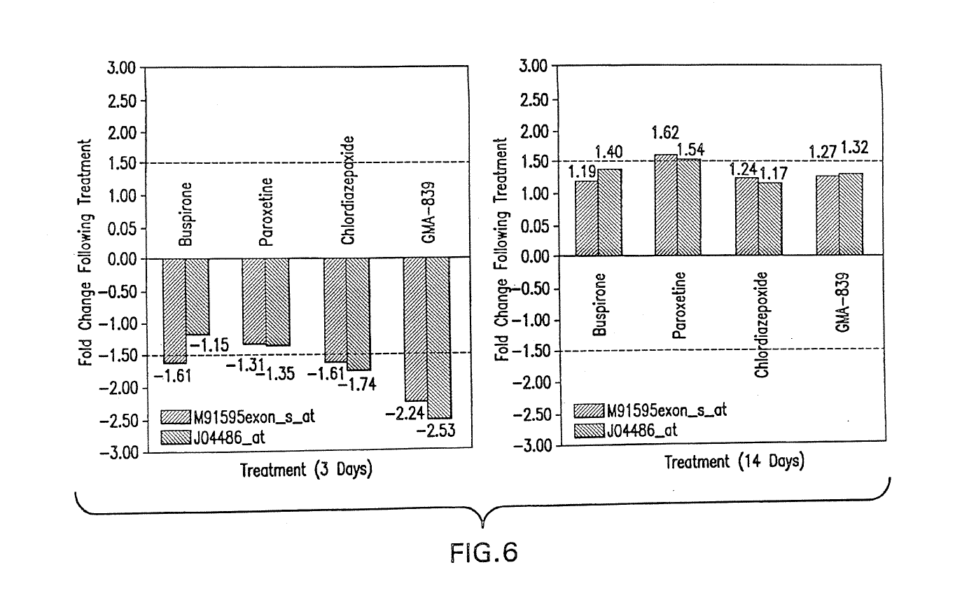

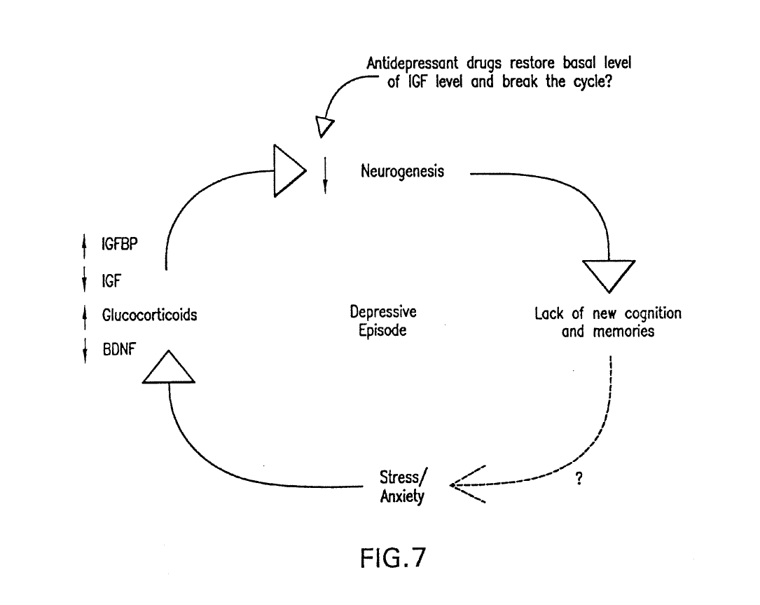

In order to learn more about the effect of venlafaxine in the brain, two dimensional gel electrophoresis patterns of hippocampal cytosolic extracts of chronic antidepressant-treated (venlafaxine; fluoxetine) and control (untreated) rats were compared quantitatively. Thirty-three spots (31 upregulated; 2 downregulated) were identified as being shared by both antidepressant drug treatments and different in integrated intensity by at least a factor of 1.5 versus control (FIG. 6). The spots were subsequently identified by mass spectrometry. The identification of several proteins suggest that venlafaxine and fluoxetine may have important functions linked to neurogenic pathways, vesicular trafficking and steroid pathway-mediated regulatory events. The findings indicate that a population of antidepressant-modulated proteins within the hippocampus includes some downstream proteins involved in complex mechanisms of action to promote the outgrowth and maintenance of neuronal processes (e.g., IGF-IA). IGF-I is initially synthesized as a 144 amino acid, inactive high molecular weight, propeptide precursor that is post-translationally processed to yield the 70 amino acid, mature peptide (Duguay et al., 1997, Steenbergh et al., 1991). IGF-II is also synthesized as a high molecular weight propeptide (Liu et al., 1993). These propeptides may also posses biological activity. These data suggest that venlafaxine and fluoxetine may have important and wide-ranging neuronal functions in the hippocampus which are beneficial to their long-term antidepressant activities in vivo (FIG. 7).

### Example 6

### IGFBP-2 mRNA Shows Altered Expression in Anxiolytic and Antidepressant-Treated Rat Amygdala

Chronic mild stress in rats causes anxiety and subsequent depression in rats (Papp et al., 1996). The same is true in human subjects (FIG. 7). This co-morbidity may share common molecular mechanisms. Antidepressant and anxiolytic drugs may ameliorate both depressed and anxious phenotypes. For example, buspirone has been shown to reverse the depressed phenotype in the rat chronic mild stress model (Papp et al., 1996).

In this experiment, rats were treated with Buspirone, Paroxetine, Chlordiazepoxide and GMA-839, drugs possessing anxiolytic and antidepressant properties, for 3 or 14 days. Transcriptional profiling of amygdala demonstrated that 3 day treatment decreased expression of IGFBP-2 mRNA across all treatments, compared to vehicle alone (FIG. 6). Conversely, 14 day treatment increased expression of IGFBP-2 mRNA across all treatments, compared to vehicle alone (FIG. 6). In the short-term 3 day treatment, it is possible that the drugs might exert their antidepressant effects through decreasing IGFBP-2 expression, which would increase bioavailability of IGF-I. The long-term treatment is of equivalent magnitude but in the opposite direction. This may represent a compensatory mechanism in response to the short-term drug effects.

### Example 7

### Small Molecules and Peptides that Prevent Formation of the Ternary Complex in the Brain

IGF-I usually exists as a ternary complex composed of IGF-I, IGFBP-3 and an acid labile subunit (ALS). IGFBP and ALS generally serves to inhibit IGF activity by reducing bioavailable IGF levels. Thus, the invention provides small molecules and/or peptides compositions to bind to the IGFBP or ALS, thus preventing or dissociating the ternary complex, thereby increasing bioavailable IGF. Since IGF-I can cross the blood brain barrier, higher levels of IGF would also be present in the brain leading to enhanced neurogenesis, amelioration of depression and the like. Binding protein-specific inhibitors may result in the release of IGF in only those tissues that contain the targeted binding proteins. For example, IGFBP-2 is more prevalent in the brain. Thus, a small molecule that is capable of crossing the blood brain barrier will release IGF in the brain.

To screen for compounds which interfere with binding of IGF and IGFBP, a Scintillation Proximity Assay can used. In this assay, IGFBP is labeled with an isotope such as ¹²⁵I. IGF is labeled with a scintillant, which emits light when proximal to radioactive decay (i.e., when IGF is bound to IGFBP). A reduction in light emission will indicate that a compound has interfered with the binding of IGF to IGFBP.

Alternatively a Fluorescence Energy Transfer (FRET) assay could be used. In a FRET assay of the invention, a fluorescence energy donor is comprised on one protein (e.g., IGFBP) and a fluorescence energy acceptor is comprised on a second protein (e.g., IGF). If the absorption spectrum of the acceptor molecule overlaps with the emission spectrum of the donor fluorophore, the fluorescent light emitted by the donor is absorbed by the acceptor. The donor molecule can be a fluorescent residue on the protein (e.g., intrinsic fluorescence such as a tryptophan or tyrosine residue), or a fluorophore which is covalently conjugated to the protein (e.g., fluorescein isothiocyanate, FITC). An appropriate donor molecule is then selected with the above acceptor/donor spectral requirements in mind.

Thus, in this example, an IGFBP is labeled with a fluorescent molecule (i.e., a donor fluorophore) and IGF is labeled with a quenching molecule (i.e., an acceptor). When IGFBP and IGF are bound, fluorescence emission will be quenched or reduced relative the IGFBP alone. Similarly, a compound which can dissociate the interaction of the IGFBP and IGF complex, will result in an increase in fluorescence emission, which indicates the compound has interfered with the binding of IGF to IGFBP.

Another assay to detect binding or dissociation of two proteins is fluorescence polarization or anisotropy. In this assay, the investigated protein (e.g., IGF) is labeled with a fluorophore with an appropriate fluorescence lifetime. The protein sample is then excited with vertically polarized light. The value of anisotropy is then calculated by determining the intensity of the horizontally and vertically polarized emission light (Gorovits and Horowitz, 1998). Next, the labeled protein (IGF) is mixed with IGFBP and ALS and the anisotropy measured again. Because fluorescence anisotropy intensity is related to the rotational freedom of the labeled protein, the more rapidly a protein rotates in solution, the smaller the anisotropy value. Thus, if the labeled IGF protein is part of a large multimeric complex (e.g., IGF-IGFBP-ALS), the IGF protein rotates more slowly in solution (relative to free, unbound IGF) and the anisotropy intensity increases (Brazil et al., 1997). Subsequently, a compound which can dissociate the interaction of the IGF-IGFBP complex, will result in a decrease in anisotropy (i.e., the labeled IGF rotates more rapidly), which indicates the compound has interfered with the binding of IGF to IGFBP.

A more traditional assay would involve labeling IGFBP with an isotope such as ¹²⁵I, incubating with IGF, then immunoprecitating of the IGF. Compounds that increase the free IGF will decrease the precipitated counts. To avoid using radioactivity, IGFBP could be labeled with an enzyme-conjugated antibody instead.

Alternatively, the IGFBP could be immobilized on the surface of an assay plate and IGF could be labeled with a radioactive tag. A rise in the number of counts would identify compounds that had interfered with binding of IGF and IGFBP.

Evaluation of binding interactions may further be performed using Biacore technology, wherein the IGF or IGFBP is bound to a micro chip, either directly by chemical modification or tethered via antibody-epitope association (e.g., antibody to the IGF), antibody directed to an epitope tag (e.g., His tagged) or fusion protein (e.g., GST). A second protein or proteins is/are then applied via flow over the “chip” and the change in signal is detected. Finally, test compounds are applied via flow over the “chip” and the change in signal is detected.

Once a series of potential compounds has been identified for a combination of IGF, IGFBP and ALS, a bioassay can be used to select the most promising candidates. For example, a cellular assay that measures cell proliferation in presence of IGF-I and IGFBP was described above. This assay could be modified to test the effectiveness of small molecules that interfere with binding of IGF and IGFBP in enhancing cellular proliferation. An increase in cell proliferation would correlate with a compound's potency.

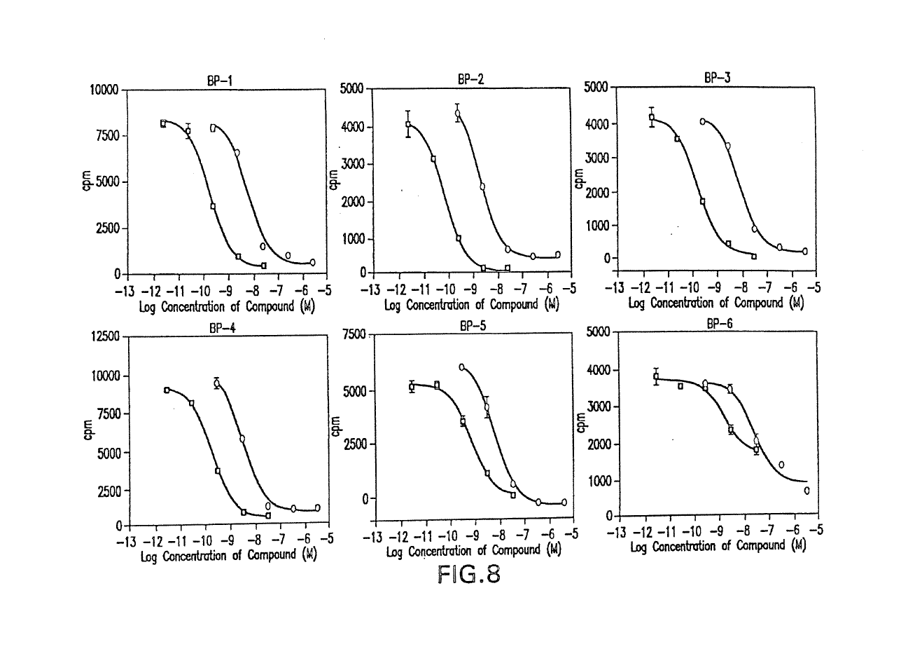

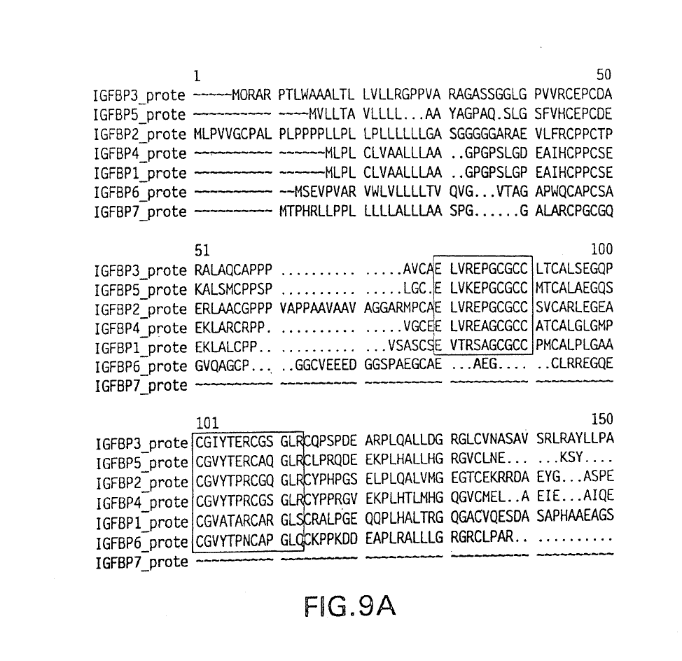

For example, it has previously been demonstrated that the isoquinoline analogue NBI-31772 dissociates IGF-I from its binding protein complex (FIG. 8) (Neurocrine Biosciences, Liu et al., 2001; Chen et al., 2001). The released IGF-I is biologically active in an in vitro fibroblast proliferation bioassay.

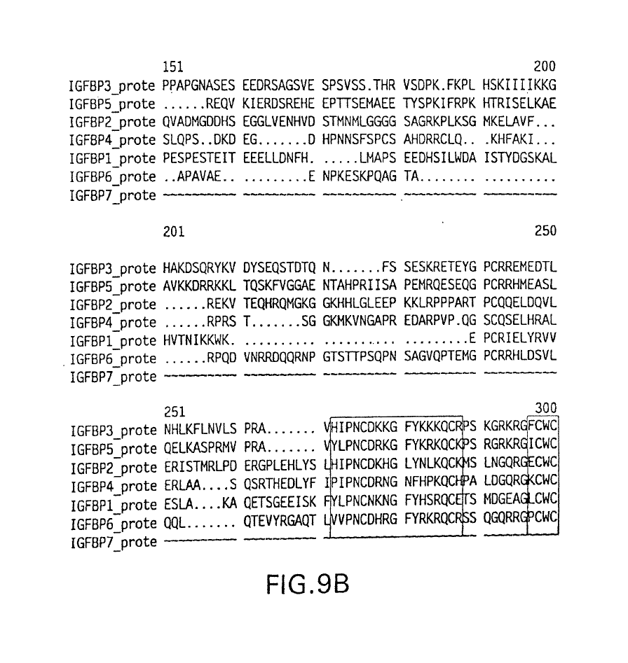

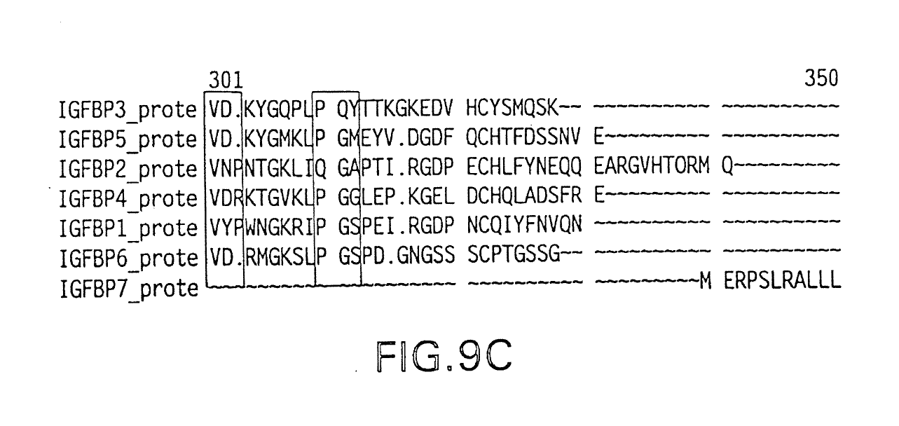

It is also known that NBI-31772 inhibits interaction of IGF-I with IGFBP-1 to 6. This is most likely due to conserved IGF binding domains on the IGFBPs. In addition, an amino acid sequence homology alignment using PileUp (Needleman and Wunsch, 1970) revealed some conserved residues across all human IGFBP family members (FIG. 9). These residues might occur at the site of binding to IGF-I.

### Example 8

### Chronic Intracerebroventricular Administration of IGF-1 Increases Proliferation in the Adult Rat Dentate Gyrus

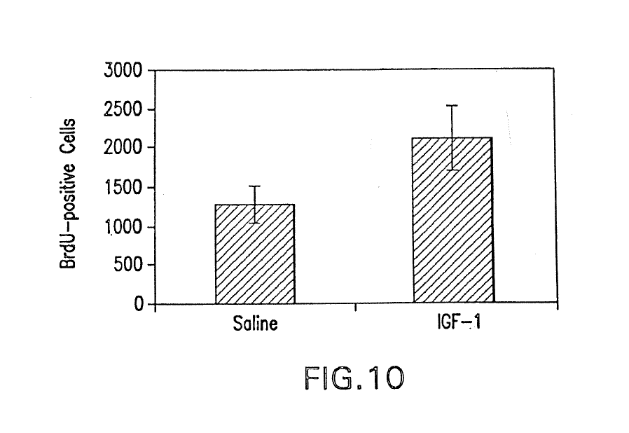

Previous investigators have shown that IGF-1 administered either intracerebroventricular (icv) or systemically increases proliferation and survival, and (Aberg, et al, 2000; Lichtenwalner et al, 2000). Furthermore, systemic IGF promotes neuronal differentiation. The present study confirms and extends these previous findings. Rats were given IGF-1 for 10 days via a cannula attached to a semiosmotic minipump. On Day 11, animals were sacrificed and quantitative analysis was performed to determine the number of BrdU-positive cells as a measure of cell proliferation. A 66% increase in BrdU-positive cells per hippocampus compared to saline-infused animals was observed (FIG. 10). This is a larger increase than is seen with the chemical antidepressants and indicates that the IGF-1 pathway may be a novel therapeutic target with which to increase proliferation or neurogenesis.

### Example 9

### Anxiolytic Effect of IGFBP Inhibition

To assess the anxiolytic activity of NBI-31772, mice receiving intraventricular administration of NBI-31772 were tested in an elevated zero maze (EZM) or in a four-plate appratus (FPT). NBI-31722 (availabe from Calbiochem/EMD Biosciences, Inc. of San Diego, Calif.) was administered in a volume of 2 μl. Balb/C mice were used for the elevated zero maze and Swiss-Webster mice were used for the four-plate test. One-way analysis of variance (ANOVA) was performed on behavioral data to determine effects of test compound treatments, followed by Least Significant Difference Tests for a post hoc analysis. Data is presented as the mean±SEM.

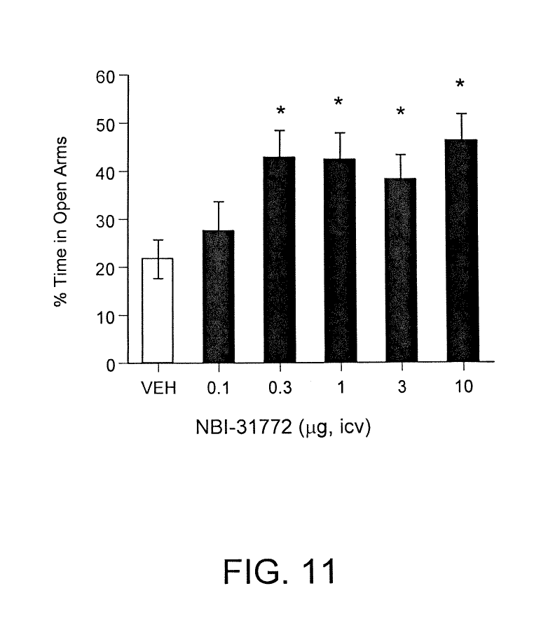

In the mouse elevated zero maze (EZM), NBI-31772 (0.1-10 μg, icv) administered 20 minutes prior to testing produced a dose-dependent increase in the % time spent in the open arms of the maze (FIG. 11; F(5,81)=3.613, p<0.05). Post-hoc analysis revealed that the 0.3-10 μg doses produced significant (p<0.05) increases in the % time in the open arms compared to the vehicle group (100% increase in the 0.3 and 1 μg group, 81% in the 3 μg group, and 119% in the 10 μg group). There was no effect on the total distance moved (cm) in the 0.1-1.0 μg groups (p>0.05), while the 3 and 10 μg doses produced significant increases in the total distance moved (p<0.01) (FIG. 11). These effects are similar to those seen with reference anxiolytic compounds chlordiazepoxide and diazepam (Ring et al., Psychopharmacology (Berl.), 2006, 185(2):218-225) and are indicative of anxiolytic-like activity of NBI-31772 in the EZM.

The four-plate apparatus consists of a Plexiglas chamber (18×25×16 cm) with a floor consisting of four rectangular metal plates (8×11 cm), which are separated from one another by a 4 mm gap. All four plates are wired to a shock generator (0.8 mA, 0.5 second; Med Associates Inc. of St. Albans, Vt.). In each experiment, mice are placed into the chamber and given an 18-second habituation period followed by a 1-minute test. During the test, the animal's innate motivation to explore the novel environment is suppressed by the delivery of a mild foot shock every time the animal moves from one plate to another (referred to as a ‘punished crossing’). Following each punished crossing, there is a 3-second time out where the mouse may cross the plates without receiving another shock. An experimenter blind to the dosing conditions administers shocks, and a computer records the total number of punished crossings an animal makes during the testing period. Clinically effective classes of anxiolytic compounds such as benzodiazepines, selective serotonin reuptake inhibitors, or 5-HT_{1A} antagonists produce increases in punished crossings, which is indicative of anxiolytic-like activity.

In each study, mice were given a single icv injection or two icv injections of test compounds or vehicle 20 minutes prior to the test session, and the number of punished crosses during the 1-minute test was recorded. For experiments where 2 icv injections were given, an animal received an injection in both the left and right ventricles, with the second injection given immediately after the first injection. A 26-gauge Hamilton syringe with 3-mm needle was used for injections and the injection site was visualized by locating the middle of the invisible line that runs diagonally from the left eye to the right ear. Mice were housed individually during the interval between injections and behavioral testing.

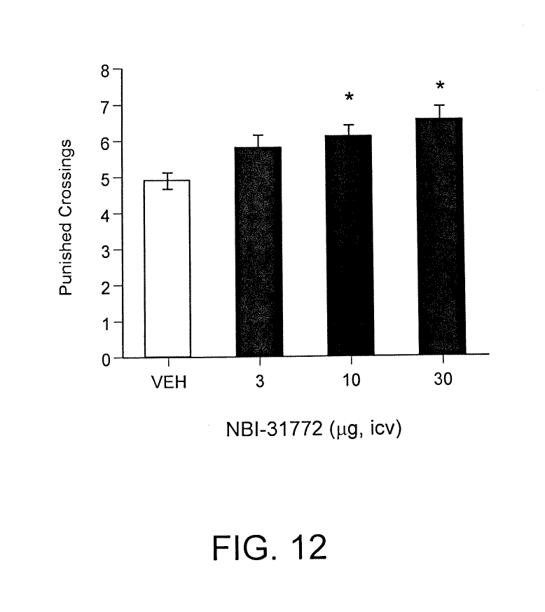

Central administration of NBI-31772 (3.0-30 μg, icv) produced a dose-dependent increase in punished crossings in this model (FIG. 12; F(5,74)=13.215, p<0.001). Post hoc analysis revealed significant increases in punished crossings at 10 and 30 μg (22% and 32% for 10 and 30 μg respectively; p<0.05). At higher doses of NBI-31772 (56 and 100 μg), an increase in punished crossings was seen although seizures were observed in a percentage of animals in both groups (20% and 40% of animals in the 56 and 100 μg group respectively) and these doses were not used for analysis. The increase in punished crossings at 10 and 30 μg suggests anxiolytic-like activity of NBI-31772.

The present invention also encompasses systemic administration of NBI-31771. While there is no published evidence that NBI-31772 crosses the blood brain barrier, it may raise circulating IGF, which then enters the brain.

### Example 10

### Anxiolytic Effect of Increased Levels of IGF-1 and IGF-1R Activation

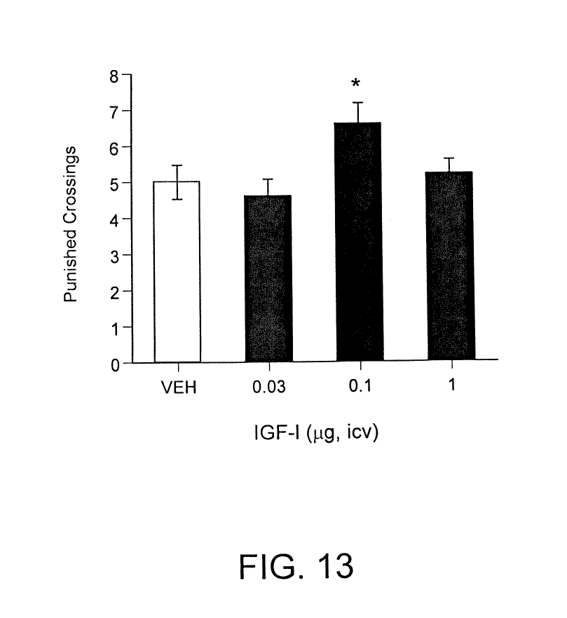

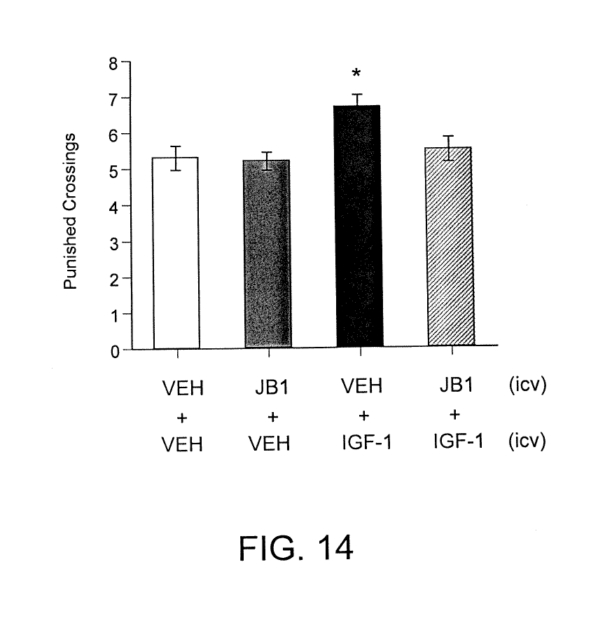

The mouse four-plate test was performed and statistical analyses conducted as described in Example 9. Central administration of IGF-1 (0.03-1.0 μg recombinant human IGF-1, available from Bachem Biosciences Inc. of King of Prussia, Pa.) 20 minutes prior to testing produced a significant overall effect on punished crossings (FIG. 13; F(3,36)=3.153, p<0.0366). Post hoc analysis revealed a significant increase in punished crossings after the 0.01 μg dose of IGF-1 (32% increase from vehicle; p<0.03). This increase in punished crossings suggests anxiolytic-like activity of IGF-1. When administered in combination with JB1 (30 μg, available from Bachem Biosciences Inc. of King of Prussia, Pa.), the anxiolytic-like effects of IGF-1 were completely blocked (FIG. 14)

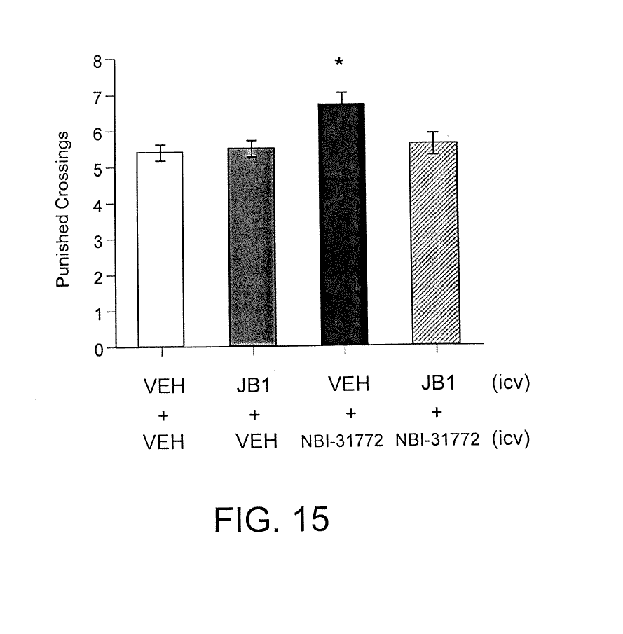

Additional studies were conducted in the mouse FPT to determine if the IGF-1R mediates the anxiolytic-like effects of NBI-31772 in a similar manner to those seen with IGF-1. In these studies, NBI-31772 and JB1, an IGF-1R antagonist, were administered centrally (icv) with the two injections occurring sequentially. Compounds were administered 20 minutes prior to testing and the number of punished crosses during the 1-minute test was recorded. Under these conditions, NBI-31772 (30 μg) increased punished crossings (FIG. 14; 24% increase compared to vehicle group; p<0.003) but when administered in combination with JB1 (10 μg), the anxiolytic-like effects of NBI-31772 were completely blocked (FIG. 15). Therefore, the anxiolytic-like effects of NBI-31772 are mediated through activation of IGF-1R. Taken together, this data shows that NBI-31772 produces its anxiolytic-like effects acts by increasing free active IGF-1 to bind to and activate the IGF-1R.

Additional models for testing antidepressant effects of IGFBP inhibition or elevated levels of IGF-1 include resident-intruder, chronic mild stress, forced swim, or the modified forced swim test developed by Irwin Lucki at the University of Pennsylvania (Cryan et al., 2002).

### Example 11

### Antidepressant Effect of IGFBP Inhibition

To assess the on potential antidepressant effects of NBI-31772, Swiss-Webster mice were analyzed using the Tail Suspension Test (TST). In this test, mice were suspended upside down by taping their tails with adhesive laboratory tape (VWR International of West Chester, Pa.) to a flat metal bar connected to a strain gauge within a sound-attenuating chamber (Med Associates Inc. of St. Albans, Vt.). The bar is positioned such that a mouse is not able to grasp the top or the sides of the chamber. The total time spent immobile during a 6-minute test session is automatically recorded. Compounds that decrease the total immobility time compared to control animals are predicted to have antidepressant-like effects. In each study, mice were given a single icv injection of test compound or vehicle 20 minute prior to the test session. Mice were housed individually during the interval between injection and behavioral testing.

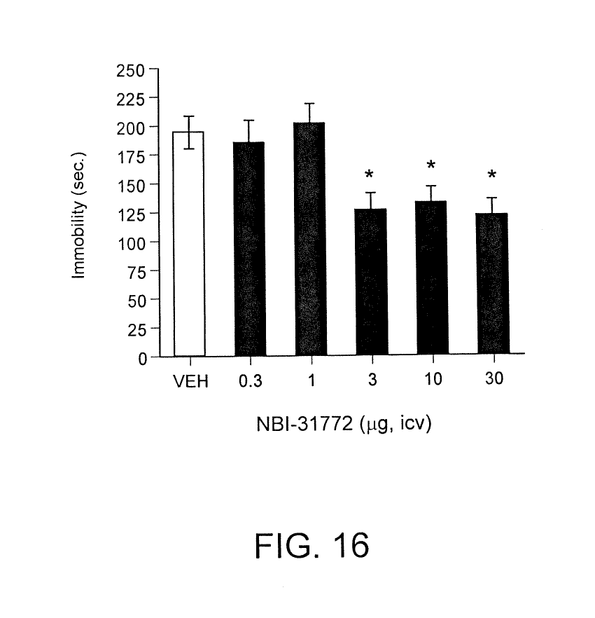

In the mouse tail suspension test, NBI-31772 (0.3-30 μg) administerd 20 minutes prior to testing produced a dose-dependent decrease in immobility (FIG. 16; F(5,70)=5.222, p<0.004). One-way analysis of variance (ANOVA) was performed on behavioral data to determine effects of NBI-31772 treatment, followed by Least Significant Difference Tests for a post hoc analysis. The data is presented as the mean±SEM. Post-hoc analysis revealed that the three highest doses (3-30 μg) produced a significant decrease in immobility (32%, 35% and 38% decrease in the 3, 10 and 30 μg groups respectively; p<0.03). This decrease in immobility indicates antidepressant-like effects similar to that seen with IGF-1.

### Example 12

### Antidepressant Effect of Increased Levels of IGF-1 and IGF-1R Activation

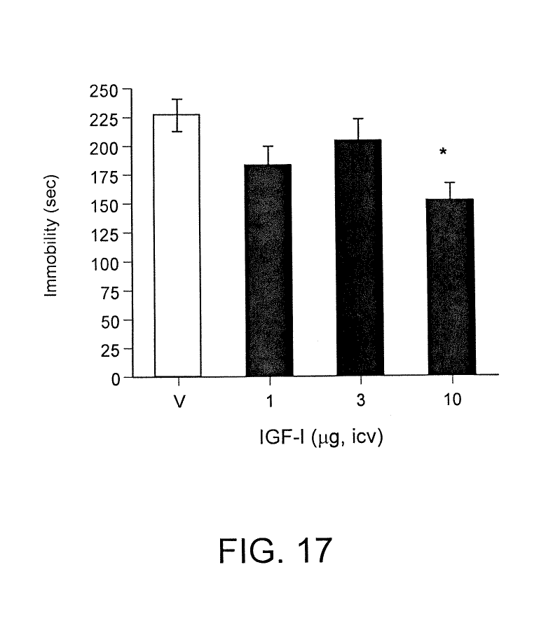

The mouse tail suspension test was performed and statistical analyses conducted as described in Example 11. In the mouse tail suspension test, IGF-1 (1-10 μg) administered 20 minutes prior to testing produced a dose-dependent decrease in immobility (FIG. 17; F(3,43)=3.71, p<0.02). Post-hoc analysis revealed that the 10 μg dose produced a significant decrease in immobility (33% decrease from vehicle; p<0.003). This decrease in immobility indicates a putative antidepressant-like effect and is in agreement with previous data demonstrating antidepressant-like activity of IGF-1 in the rat (Hosahw et al., Brain Res., 2005, 1037(1-2):204-208).

### Example 13

### Identifying Selective IGFBP Targets

It is contemplated in particular embodiments to design a drug which displaces IGF from a specific binding protein (e.g., IGFBP or ALS) and is targetable to a binding protein which shows tissue-predominant expression. In one example, recombinant variants of IGF-I have been produced which lose their affinity of IGFBP-1 yet retained their affinity for IGFBP-3, thus indicating that different domains of the IGF molecule bind to different IGFBP (Dubaquie and Lowman 1999; Dubaquie et al., 2001).

The highest activity of the isoquinoline analogue NBI-31772 is toward IGFBP-2, compared to the other five IGFBPs. Without being bound to any particular mode of action, NBI-31772 may ameliorate depression and/or anxiety by displacing IGF-1 IGFBP-2.

### Example 14

### Determination of the Bioactivity of IGF, IGFBP, and ALS Combinations on Neuronal Cells

One means of performing a systematic survey of the biological activities of IGF molecules on neuronal cells could be to determine whether binding of IGF and IGFBP increase or decrease proliferation of cells. Combinations of IFG-I or IGF-II, IGFBP-1 to 7, and ALS (there are 24 total combinations) are tested for their mitogenic ability in a cell culture system. Cultured neural cells, or alternatively, cells known to be responsive to IGF (e.g., fibroblasts) also are tested. Cell proliferation is tested by incorporation of tritiated thymidine, with the goal of identifying combinations of IGF, IGFBP and ALS that inhibit cell proliferation, compared to IGF alone.

### Example 15

### Histological and Behavioral Tests on IGF Transgenics and Knockouts

Transgenic and knockout animals have been generated for most IGF-I, IGF-II and IGFBPs. BRDU labeling of dividing cells in the dentate gyrus, with co-staining for neuronal markers, can be used to determine whether these animals show enhanced or diminished neurogenesis.

Transgenic and knockout animals could also be tested for enhanced or diminished activity in behavioral models which test for a depressed or anhedonic phenotype. The behavioral despair model (forced swim test) can test for helplessness, which is a marker of depression. Since neurogenesis is a consequence of learning (Gould et al 1999) and may be a requirement for learning (Shors et al 2001), the ability of such transgenic and knockout animals to learn can also be tested.

### Example 16

### Inhibition of IGFBP Expression

Design of RNA Molecules as Compositions of the Invention. All RNA molecules in this experiment are approximately 600 nucleotides in length, and all RNA molecules are designed to be incapable of producing functional IGFBP protein. The molecules have no cap and no poly-A sequence; the native initiation codon is not present, and the RNA does not encode the full-length product. The following RNA molecules are designed:
(1) a single-stranded (ss) sense RNA polynucleotide sequence homologous to a portion of IGFBP messenger RNA (mRNA);
(2) a ss anti-sense RNA polynucleotide sequence complementary to a portion of IGFBP mRNA,
(3) a double-stranded (ds) RNA molecule comprised of both sense and anti-sense to a portion of IGFBP mRNA polynucleotide sequences,
(4) a ss sense RNA polynucleotide sequence homologous to a portion of IGFBP heterogeneous RNA (hnRNA),
(5) a ss anti-sense RNA polynucleotide sequence complementary to a portion of IGFBP hnRNA,
(6) a ds RNA molecule comprised of the sense and anti-sense IGFBP hnRNA polynucleotide sequences,
(7) a ss RNA polynucleotide sequence homologous to the top strand of the portion of the IGFBP promoter,
(8) a ss RNA polynucleotide sequence homologous to the bottom strand of the portion of the IGFBP promoter, and
(9) a ds RNA molecule comprised of RNA polyriucleotide sequences homologous to the top and bottom strands of the IGFBP promoter.

The various RNA molecules of (1)-(9) above may be generated through T7 RNA polymerase transcription of PCR products bearing a T7 promoter at one end. In the instance where a sense RNA is desired, a T7 promoter is located at the 5′ end of the forward PCR primer. In the instance where an antisense RNA is desired, the T7 promoter is located at the 5′ end of the reverse PCR primer. When dsRNA is desired, both types of PCR products may be included in the T7 transcription reaction. Alternatively, sense and anti-sense RNA may be mixed together after transcription, under annealing conditions, to form ds RNA.

Assay. Balb/c mice (5 mice/group) may be injected intercranially with the IGFBP chain specific RNAs described above or with controls at doses ranging between 10 μg and 500 μg. Brains are harvested from a sample of the mice every four days for a period of three weeks and assayed for IGFBP levels using antibodies or by northern blot analysis for reduced RNA levels.

### Example 17

### Antisense Inhibition of IGFBP Expression

Antisense preparation can be performed using standard techniques including the use of kits such as those of Sequitur Inc. (Natick, Mass.). The following procedure utilizes phosphorothioate oligodeoxynucleotides and cationic lipids. The oligomers are selected to be complementary to the 5′ end of the mRNA so that the translation start site is encompassed.
1) Prior to plating the cells, the walls of the plate are gelatin coated to promote adhesion by incubating 0.2% sterile filtered gelatin for 30 minutes and then washing once with PBS. Cells are grown to 40-80% confluence. Hela cells can be used as a positive control.
2) The cells are washed with serum free media (such as Opti-MEMA from Gibco-BRL).
3) Suitable cationic lipids (such as Oligofectibn A from Sequitur, Inc.) are mixed and added to serum free media without antibiotics in a polystyrene tube. The concentration of the lipids can be varied depending on their source. Add oligomers to the tubes containing serum free media/cationic lipids to a final concentration of approximately 200 nM (50-400 nM range) from a 1001M stock (2 μl per ml) and mix by inverting.
4) The oligomer/media/cationic lipid solution is added to the cells (approximately 0.5 mL for each well of a 24 well plate) and incubated at 37° C. for 4 hours.
5) The cells are gently washed with media and complete growth media is added. The cells are grown for 24 hours. A certain percentage of the cells may lift off the plate or become lysed.
6) Cells are harvested and IGFBP gene expression is measured.

Equivalents: Those skilled in the art will recognize, or be able to ascertain using no more than routine experimentation, many equivalents to the specific embodiments of the invention described herein. Such equivalents are intended to be encompassed by the following claims.

What is claimed is:

1. A method of screening for a neurological disorder in a human subject comprising the steps of:
(a) obtaining a biological sample from the subject;
(b) contacting the sample with a polynucleotide probe complementary to an IGFBP-2 mRNA;
(c) measuring the amount of probe bound to the mRNA;
(d) comparing the amount in step (c) with IGFBP-2 mRNA in human samples obtained from a statistically significant population lacking the neurological disorder,
wherein higher IGFBP-2 levels in the subject indicates a predisposition to the neurological disorder.

2. The method of claim 1, wherein the neurological disorder is selected from the group consisting of depression, anxiety, panic disorder, bipolar disorder, insomnia, obsessive compulsive disorder, dysthymic disorder and schizophrenia.

3. The method of claim 1, wherein the biological sample is obtained as a blood sample, a cerebrospinal fluid (CSF) sample, a saliva sample, a skin biopsy or a buccal biopsy.

4. The method of claim 1, wherein the biological sample is selected from the group consisting of blood plasma, serum, erythrocytes, leukocytes, platelets, lymphocytes, macrophages, fibroblast cells, mast cells, fat cells and epithelial cells.

5. The method of claim 1, wherein the probe comprises a nucleotide sequence which hybridizes under high stringency hybridization conditions with a polynucleotide comprising the nucleotide sequence of SEQ ID NO:8.

6. A method for treating a neurological disorder in a human in need thereof the method comprising administering to the human a therapeutically effective amount of a composition which dissociates a protein complex comprising an Insulin-like growth factor (IGF) and an Insulin-like growth factor binding protein (IGFBP).

7. The method of claim 6, wherein the protein complex is further defined as a dimeric complex comprising IGF and IGFBP.

8. The method of claim 7, wherein the protein complex further comprises an acid labile subunit (ALS), wherein the ratio of IGF to IGFBP to ALS is 1:1:1.

9. The method of claim 6, wherein the composition crosses the blood brain barrier

10. The method of claim 6, wherein the composition is a small molecule.

11. The method of claim 6, wherein the composition is a peptide.

12. The method of claim 6, wherein the composition is a peptide mimetic.

13. The method of claim 6, wherein the composition is an antisense molecule which inhibits expression of an IGBFP.

14. The method of claim 6, wherein the neurological disorder is selected from the group consisting of depression, anxiety, panic disorder, bi-polar disorder, insomnia, obsessive compulsive disorder, dysthymic disorder and schizophrenia.

15. The method of claim 6, wherein the protein complex is comprised in the central nervous system (CNS).

16. The method of claim 15, wherein the CNS is further defined as the brain.

17. The method of claim 16, wherein the brain is further defined as a region of the brain selected from the group consisting of the dentate gyrus, the hippocampus; the subventricular zone and the cortex.

18. The method of claim 6, wherein the IGFBP is IGFBP-1, IGFBP-2, or IGFBP-5.

19. The method of claim 6, wherein the IGF is IGF-I.

20. The method of claim 6, wherein the IGF is IGF-II.

21. An antisense RNA molecule which inhibits the expression of an IGFBP.

22. The RNA molecule of claim 21, wherein the molecule is antisense to a polynucleotide having a nucleotide sequence of SEQ ID NO:8 or a degenerate variant thereof.

23. A pharmaceutical composition which dissociates a protein complex comprising an Insulin-like growth factor (IGF) and an Insulin-like growth factor binding protein (IGFBP).

24. The composition of claim 23, wherein the protein complex is further defined as a dimeric complex comprising IGF and IGFBP.

25. The composition of claim 24, wherein the protein complex further comprises an acid labile subunit (ALS), wherein the ratio of IGF to IGFBP to ALS is 1:1:1.

26. The composition of claim 24, wherein the composition is a small molecule.

27. The composition of claim 24, wherein the composition is a peptide.

28. A method of screening for compounds which dissociate an IGF/IGFBP/ALS trimer complex, the method comprising:
(a) providing a sample comprising an IGF polypeptide, an IGFBP polypeptide and an ALS polypeptide, wherein the IGFBP is labeled with a radioactive isotope and the IGF is labeled with a scintillant,
(b) contacting the sample with a test compound; and
(c) detecting light emission of the scintillant,
wherein a reduction in light emission, relative to a sample in the absence of the test compound, indicates a test compound which dissociates the complex.

29. A method of screening for compounds which dissociate an IGF/IGFBP/ALS trimer complex, the method comprising:
(a) providing a sample comprising an IGF polypeptide, an IGFBP polypeptide and an ALS polypeptide, wherein the IGFBP is labeled with a radioactive isotope;
(b) contacting the sample with a test compound,
(c) immunoprecipitating the sample with an anti-IGF antibody; and
(d) measuring the radioactivity of the precipitate,
wherein a reduction in radioactivity, relative to a sample in the absence of the test compound, indicates a test compound which dissociates the complex.

30. A method of screening for compounds which dissociate an IGF/IGFBP/ALS trimer complex, the method comprising:
(a) providing a sample comprising an IGF polypeptide, an IGFBP polypeptide and an ALS polypeptide, wherein the IGFBP is labeled with a fluorescence donor molecule and the IGF is labeled with a fluorescence acceptor molecule,
(b) contacting the sample with a test compound,
(c) exciting the sample at the excitation wavelength of the donor molecule; and
(d) detecting fluorescence at the emission wavelength of the acceptor molecule,
wherein a fluorescence signal, relative to a sample in the absence of the test compound, indicates a test compound which dissociates the complex.

31. A method of screening for compounds which dissociate an IGF/IGFBP/ALS trimer complex, the method comprising:
(a) providing a sample comprising an IGF polypeptide, an IGFBP polypeptide and an ALS polypeptide, wherein the IGF is labeled with a fluorophore,
(b) contacting the sample with a test compound,
(c) exciting the fluorophore at its excitation wavelength; and
(d) detecting the fluorescence polarization of fluorophore,
wherein a decrease in polarization, relative to a sample in the absence of the test compound, indicates a test compound which dissociates the complex.

32. A method of treating depression in a subject comprising administering an agent that results in activation of IGF-1R in the brain of the subject.

33. The method of claim 32, wherein the agent elevates levels of IGF-1 in the brain of the subject.

34. A method of treating depression in a subject comprising administering to a subject an effective amount of an IGFBP inhibitor.

35. The method of claim 34, wherein the IGFBP inhibitor disrupts a protein complex comprising IGF and IGFBP.

36. The method of claim 35, wherein the IGFBP inhibitor disrupts a protein complex comprising IGF, IGFBP, and an acid labile subunit (ALS).

37. The method of claim 34, wherein the IGF is IGF-I.

38. The method of claim 34, wherein the IGFBP inhibitor crosses the blood brain barrier.

39. The method of claim 34, wherein the IGFBP inhibitor is administered directly to the central nervous system (CNS).

40. The method of claim 39, wherein the IGFBP inhibitor is administered intrathecally, intraventricularly, or other means for direct brain administration.

41. The method of claim 39, wherein the IGFBP inhibitor is a nonpeptide small molecule.

42. The method of claim 41, wherein the nonpeptide small molecule is an isoquinoline analogue.

43. The method of claim 42, wherein the isoquinoline analogue is NBI-31772 (1-(3,4-dihydroxybenzoyl)-3-hydroxy-carbonyl-6,7-dihydroxy-isoquinoline).

44. The method of claim 34, wherein the IGFBP inhibitor is a peptide, a peptide mimetic, or an antisense molecule which inhibits expression of an IGFBP.

45. A method of treating anxiety in a subject comprising administering an agent that results in activation of IGF-1R in the brain of the subject.

46. The method of claim 45, wherein the agent elevates levels of IGF-1 in the brain of the subject.

47. A method of treating anxiety in a subject comprising administering to a subject an effective amount of an IGFBP inhibitor.

48. The method of claim 47, wherein the IGFBP inhibitor disrupts a protein complex comprising IGF and IGFBP.

49. The method of claim 48, wherein the IGFBP inhibitor disrupts a protein complex comprising IGF, IGFBP, and an acid labile subunit (ALS).

50. The method of claim 45, wherein the IGF is IGF-I.

51. The method of claim 45, wherein the IGFBP inhibitor crosses the blood brain barrier.

52. The method of claim 45, wherein the IGFBP inhibitor is administered directly to the central nervous system (CNS).

53. The method of claim 52, wherein the IGFBP inhibitor is administered intrathecally, intraventricularly, or other means for direct brain administration.

54. The method of claim 45, wherein the IGFBP inhibitor is a nonpeptide small molecule.

55. The method of claim 54, wherein the nonpeptide small molecule is an isoquinoline analogue.

56. The method of claim 55, wherein the isoquinoline analogue is NBI-31772 (1-(3,4-dihydroxybenzoyl)-3-hydroxy-carbonyl-6,7-dihydroxy-isoquinoline).

57. The method of claim 45, wherein the IGFBP inhibitor is a peptide, a peptide mimetic, or an antisense molecule which inhibits expression of an IGFBP.

58. A method of elevating IGF-1 levels in the brain of a subject comprising administering to the subject an effective amount of an IGFBP inhibitor.

59. The method of claim 58, wherein the IGFBP inhibitor disrupts a protein complex comprising IGF and IGFBP.

60. The method of claim 59, wherein the IGFBP inhibitor disrupts a protein complex comprising IGF, IGFBP, and an acid labile subunit (ALS).

61. The method of claim 58, wherein the IGFBP inhibitor crosses the blood brain barrier.

62. The method of claim 58, wherein the IGFBP inhibitor is administered directly to the central nervous system (CNS).

63. The method of claim 62, wherein the IGFBP inhibitor is administered intrathecally, intraventricularly, or other means for direct brain administration.

64. The method of claim 63, wherein the IGFBP inhibitor is a nonpeptide small molecule.

65. The method of claim 64, wherein the nonpeptide small molecule is an isoquinoline analogue.

66. The method of claim 65, wherein the isoquinoline analogue is NBI-31772 (1-(3,4-dihydroxybenzoyl)-3-hydroxy-carbonyl-6,7-dihydroxy-isoquinoline).


SEQUENCE LISTING (21 sequences)

SEQ ID NO: 1 - DNA, 7260 bp, Homo sapiens
tcactgtcactgctaaattcagagcagattagagcctgcgcaatggaataaagtcctcaaaattgaaatgtgacattgctctcaacatctcccatctctctggatttccttttgcttcattattcctgctaaccaattcattttcagactttgtacttcagaagcaatgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatgtaggaagaccctcctgaggagtgaagagtgacatgccaccgcaggatcctttgctctgcacgagttacctgttaaactttggaacacctaccaaaaaataagtttgataacatttaaaagatgggcgtttcccccaatgaaatacacaagtaaacattccaacattgtctttaggagtgatttgcaccttgcaaaaatggtcctggagttggtagattgctgttgatcttttatcaataatgttctatagaaaagaaaaaaaaatatatatatatatatatcttagtccctgcctctcaagagccacaaatgcatgggtgttgtatagatccagttgcactaaattcctctctgaatcttggctgctggagccattcattcagcaaccttgtctaagtggtttatgaattgtttccttatttgcacttctttctacacaactcgggctgtttgttttacagtgtctgataatcttgttagtctatacccaccacctcccttcataacctttatatttgccgaatttggcctcctcaaaagcagcagcaagtcgtcaagaagcacaccaattctaacccacaagattccatctgtggcatttgtaccaaatataagttggatgcattttattttagacacaaagctttatttttccacatcatgcttacaaaaaagaataatgcaaatagttgcaactttgaggccaatcatttttaggcatatgttttaaacatagaaagtttcttcaactcaaaagagttccttcaaatgatgagttaatgtgcaacctaattagtaactttcctctttttattttttccatatagagcactatgtaaatttagcatatcaattatacaggatatatcaaacagtatgtaaaactctgttttttagtataatggtgctattttgtagtttgttatatgaaagagtctggccaaaacggtaatacgtgaaagcaaaacaataggggaagcctggagccaaagatgacacaaggggaagggtactgaaaacaccatccatttgggaaagaaggcaaagtccccccagttatgccttccaagaggaacttcagacacaaaagtccactgatgcaaattggactggcgagtccagagaggaaactgtggaatggaaaaagcagaaggctaggaattttagcagtcctggtttctttttctcatggaagaaatgaacatctgccagctgtgtcatggactcaccactgtgtgaccttgggcaagtcacttcacctctctgtgcctcagtttcctcatctgcaaaatgggggcaatatgtcatctacctacctcaaaggggtggtataaggtttaaaaagataaagattcagatttttttaccctgggttgctgtaagggtgcaacatcagggcgcttgagttgctgagatgcaaggaattctataaataacccattcatagcatagctagagattggtgaattgaatgctcctgacatctcagttcttgtcagtgaagctatccaaataactggccaactagttgttaaaagctaacagctcaatctcttaaaacacttttcaaaatatgtgggaagcatttgattttcaatttgattttgaattctgcatttggttttatgaatacaaagataagtgaaaagagagaaaggaaaagaaaaaggagaaaaacaaagagatttctaccagtgaaaggggaattaattactctttgttagcactcactgactcttctatgcagttactacatatctagtaaaaccttgtttaatactataaataatattctattcattttgaaaaacacaatgattccttcttttctaggcaatataaggaaagtgatccaaaatttgaaatattaaaataatatctaataaaaagtcacaaagttatcttctttaacaaactttactcttattcttagctgtatatacatttttttaaaaagtttgttaaaatatgcttgactagagtttcagttgaaaggcaaaaacttccatcacaacaagaaatttcccatgcctgctcagaagggtagcccctagctctctgtgaatgtgttttatccattcaactgaaaattggtatcaagaaagtccactggttagtgtactagtccatcatagcctagaaaatgatccctatctgcagatcaagattttctcattagaacaatgaattatccagcattcagatctttctagtcaccttagaactttttggttaaaagtacccaggcttgattatttcatgcaaattctatattttacattcttggaaagtctatatgaaaaacaaaaataacatcttcagtttttctcccactgggtcacctcaaggatcagaggccaggaaaaaaaaaaaagactccctggatctctgaatatatgcaaaaagaaggccccatttagtggagccagcaatcctgttcagtcaacaagtattttaactctcagtccaacattatttgaattgagcacctcaagcatgcttagcaatgttctaatcactatggacagatgtaaaagaaactatacatcatttttgccctctgcctgttttccagacatacaggttctgtggaataagatactggactcctcttcccaagatggcacttctttttatttcttgtccccagtgtgtaccttttaaaattattccctctcaacaaaactttataggcagtcttctgcagacttaacatgttttctgtcatagttagatgtgataattctaagagtgtctatgacttatttccttcacttaattctatccacagtcaaaaatcccccaaggaggaaagctgaaagatgcaactgccaatattatctttcttaactttttccaacacataatcctctccaactggattataaataaattgaaaataactcattataccaattcactattttattttttaatgaattaaaactagaaaacaaattgatgcaaaccctggaagtcagttgattactatatactacagcagaatgactcagatttcatagaaaggagcaaccaaaatgtcacaaccaaaactttacaagctttgcttcagaattagattgctttataattcttgaatgaggcaatttcaagatatttgtaaaagaacagtaaacattggtaagaatgagctttcaactcataggcttatttccaatttaattgaccatactggatacttaggtcaaatttctgttctctcttgcccaaataatattaaagtattatttgaactttttaagatgaggcagttcccctgaaaaagttaatgcagctctccatcagaatccactcttctagggatatgaaaatctcttaacacccaccctacatacacagacacacacacacacacacacacacacacacacacacacacattcaccctaaggatccaatggaatactgaaaagaaatcacttccttgaaaattttattaaaaaacaaacaaacaaacaaaaagcctgtccacccttgagaatccttcctctccttggaacgtcaatgtttgtgtagatgaaaccatctcatgctctgtggctccagggtttctgttactattttatgcacttgggagaaggcttagaataaaagatgtagcacattttgctttcccatttattgtttggccagctatgccaatgtggtgctattgtttctttaagaaagtacttgactaaaaaaaaaagaaaaaaagaaaaaaaagaaagcatagacatatttttttaaagtataaaaacaacaattctatagatagatggcttaataaaatagcattaggtctatctagccaccaccacctttcaactttttatcactcacaagtagtgtactgttcaccaaattgtgaatttgggggtgcaggggcaggagttggaaattttttaaagttagaaggctccattgttttgttggctctcaaacttagcaaaattagcaatatattatccaatcttctgaacttgatcaagagcatggagaataaacgcgggaaaaaagatcttataggcaaatagaagaatttaaaagataagtaagttccttattgatttttgtgcactctgctctaaaacagatattcagcaagtggagaaaataagaacaaagagaaaaaatacatagatttacctgcaaaaaatagcttctgccaaatcccccttgggtattctttggcatttactggtttatagaagacattctcccttcacccagacatctcaaagagcagtagctctcatgaaaagcaatcactgatctcatttgggaaatgttggaaagtatttccttatgagatgggggttatctactgataaagaaagaatttatgagaaattgttgaaagagatggctaacaatctgtgaagattttttgtttcttggttttgtttttttttttttttttactttatacagtctttatgaatttcttaatgttcaaaatgacttggttcttttcttcttttttttatatcagaatgaggaataataagttaaacccacatagactctttaaaactataggctagatagaaatgtatgtttgacttgttgaagctataatcagactatttaaaatgttttgctatttttaatcttaaaagattgtgctaatttattagagcagaacctgtttggctctcctcagaagaaagaatctttccattcaaatcacatggctttccaccaatattttcaaaagataaatctgatttatgcaatggcatcatttattttaaaacagaagaattgtgaaagtttatgcccctcccttgcaaagaccataaagtccagatctggtaggggggcaacaacaaaaggaaaatgttgttgattcttggttttggattttgttttgttttcaatgctagtgtttaatcctgtagtacatatttgcttattgctattttaatattttataagaccttcctgttaggtattagaaagtgatacatagatatcttttttgtgtaatttctatttaaaaaagagagaagactgtcagaagctttaagtgcatatggtacaggataaagatatcaatttaaataaccaattcctatctggaacaatgcttttgttttttaaagaaacctctcacagataagacagaggcccaggggatttttgaagctgtctttattctgcccccatcccaacccagcccttattattttagtatctgcctcagaattttatagagggctgaccaagctgaaactctagaattaaaggaacctcactgaaaacatatatttcacgtgttccctctcttttttttcctttttgtgagatggggtctcgcactgtcccccaggctggagtgcagtggcatgatctcggctcactgcaacctccacctcctgggtttaagcgattctcctgcctcagcctcctgagtagctgggattacaggcacccaccactatgcccggctaattttttggatttttaatagagacggggttttaccatgttggccaggttggactcaaactcctgaccttgtgatttgcccgcctcagcctcccaaattgctgggattacaggcatgagccaccacaccctgcccatgtgttccctcttaatgtatgattacatggatcttaaacatgatccttctctcctcattcttcaactatctttgatggggtctttcaaggggaaaaaaatccaagcttttttaaagtaaaaaaaaaaaaagagaggacacaaaaccaaatgttactgctcaactgaaatatgagttaagatggagacagagtttctcctaataaccggagctgaattacctttcactttcaaaaacatgaccttccacaatccttagaatctgcctttttttatattactgaggcctaaaagtaaacattactcattttattttgcccaaaatgcactgatgtaaagtaggaaaaataaaaacagagctctaaaatccctttcaagccacccattgaccccactcaccaactcatagcaaagtcacttctgttaatcccttaatctgattttgtttggatatttatcttgtacccgctgctaaacacactgcaggagggactctgaaacctcaagctgtctacttacatcttttatctgtgtctgtgtatcatgaaaatgtctattcaaaatatcaaaacctttcaaatatcacgcagcttatattcagtttacataaaggccccaaataccatgtcagatctttttggtaaaagagttaatgaactatgagaattgggattacatcatgtattttgcctcatgtatttttatcacacttataggccaagtgtgataaataaacttacagacactgaattaatttcccctgctactttgaaaccagaaaataatgactggccattcgttacatctgtcttagttgaaaagcatattttttattaaattaattctgattgtatttgaaattattattcaattcacttatggcagaggaatatcaatcctaatgacttctaaaaatgtaactaattgaatcattatcttacatttactgtttaataagcatattttgaaaatgtatggctagagtgtcataataaaatggtatatctttctttagtaattacaaaaaaaaaaaaaaaaaaaaaaaa

SEQ ID NO: 2 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 3 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 4 - DNA, 1356 bp, Homo sapiens
ttctcccgcaaccttcccttcgctccctcccgtcccccccagctcctagcctccgactccctccccccctcacgcccgccctctcgccttcgccgaaccaaagtggattaattacacgctttctgtttctctccgtgctgttctctcccgctgtgcgcctgcccgcctctcgctgtcctctctccccctcgccctctcttcggcccccccctttcacgttcactctgtctctcccactatctctgcccccctctatccttgatacaacagctgacctcatttcccgataccttttcccccccgaaaagtacaacatctggcccgccccagcccgaagacagcccgtcctccctggacaatcagacgaattctccccccccccccaaaaaaaaaagccatccccccgctctgccccgtcgcacattcggcccccgcgactcggccagagcggcgctggcagaggagtgtccggcaggagggccaacgcccgctgttcggtttgcgacacgcagcagggaggtgggcggcagcgtcgccggcttccagacaccaatgggaatcccaatggggaagtcgatgctggtgcttctcaccttcttggccttcgcctcgtgctgcattgctgcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagagggacgtgtcgacccctccgaccgtgcttccggacaacttccccagataccccgtgggcaagttcttccaatatgacacctggaagcagtccacccagcgcctgcgcaggggcctgcctgccctcctgcgtgcccgccggggtcacgtgctcgccaaggagctcgaggcgttcagggaggccaaacgtcaccgtcccctgattgctctacccacccaagaccccgcccacgggggcgcccccccagagatggccagcaatcggaagtgagcaaaactgccgcaagtctgcagcccggcgccaccatcctgcagcctcctcctgaccacggacgtttccatcaggttccatcccgaaaatctctcggttccacgtccccctggggcttctcctgacccagtccccgtgccccgcctccccgaaacaggctactctcctcggccccctccatcgggctgaggaagcacagcagcatcttcaaacatgtacaaaatcgattggctttaaacacccttcacataccctcccccc

SEQ ID NO: 5 - protein, 180 aa, Homo sapiens
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 6 - DNA, 1514 bp, Homo sapiens
atcggccaccgccatcccatccagcgagcatctgccgccgcgccgccgccaccctcccagagagcactggccaccgctccaccatcacttgcccagagtttgggccaccgcccgccgccaccagcccagagagcatcggcccctgtctgctgctcgcgcctggagatgtcagaggtccccgttgctcgcgtctggctggtactgctcctgctgactgtccaggtcggcgtgacagccggcgctccgtggcagtgcgcgccctgctccgccgagaagctcgcgctctgcccgccggtgtccgcctcgtgctcggaggtcacccggtccgccggctgcggctgttgcccgatgtgcgccctgcctctgggcgccgcgtgcggcgtggcgactgcacgctgcgcccggggactcagttgccgcgcgctgccgggggagcagcaacctctgcacgccctcacccgcggccaaggcgcctgcgtgcaggagtctgacgcctccgctccccatgctgcagaggcagggagccctgaaagcccagagagcacggagataactgaggaggagctcctggataatttccatctgatggccccttctgaagaggatcattccatcctttgggacgccatcagtacctatgatggctcgaaggctctccatgtcaccaacatcaaaaaatggaaggagccctgccgaatagaactctacagagtcgtagagagtttagccaaggcacaggagacatcaggagaagaaatttccaaattttacctgccaaactgcaacaagaatggattttatcacagcagacagtgtgagacatccatggatggagaggcgggactctgctggtgcgtctacccttggaatgggaagaggatccctgggtctccagagatcaggggagaccccaactgccagatatattttaatgtacaaaactgaaaccagatgaaataatgttctgtcacgtgaaatatttaagtatatagtatatttatactctagaacatgcacatttatatatatatgtatatgtatatatatatagtaactactttttatactccatacataacttgatatagaaagctgtttatttattcactgtaagtttattttttctacacagtaaaaacttgtactatgttaataacttgtcctatgtcaatttgtatatcatgaaacacttctcatcatattgtatgtaagtaattgcatttctgctcttccaaagctcctgcgtctgtttttaaagagcatggaaaaatactgcctagaaaatgcaaaatgaaataagagagagtagtttttcagctagtttgaaggaggacggttaacttgtatattccaccattcacatttgatgtacatgtgtagggaaagttaaaagtgttgattacataatcaaagctacctgtggtgatgttgccacctgttaaaatgtacactggatatgttgttaaacacgtgtcgataatggaaacatttacaataaatattctgcatggaaa

SEQ ID NO: 7 - protein, 259 aa, Homo sapiens
MSEVPVARVWLVLLLLTVQVGVTAGAPWQCAPCSAEKLALCPPVSASCSEVTRSAGCGCCPMCALPLGAACGVATARCARGLSCRALPGEQQPLHALTRGQGACVQESDASAPHAAEAGSPESPESTEITEEELLDNFHLMAPSEEDHSILWDAISTYDGSKALHVTNIKKWKEPCRIELYRVVESLAKAQETSGEEISKFYLPNCNKNGFYHSRQCETSMDGEAGLCWCVYPWNGKRIPGSPEIRGDPNCQIYFNVQN

SEQ ID NO: 8 - DNA, 1433 bp, Homo sapiens
attcggggcgagggaggaggaagaagcggaggaggcggctcccgctcgcagggccgtgcacctgcccgcccgcccgctcgctcgctcgcccgccgcgccgcgctgccgaccgccagcatgctgccgagagtgggctgccccgcgctgccgctgccgccgccgccgctgctgccgctgctgccgctgctgctgctgctactgggcgcgagtggcggcggcggcggggcgcgcgcggaggtgctgttccgctgcccgccctgcacacccgagcgcctggccgcctgcgggcccccgccggttgcgccgcccgccgcggtggccgcagtggccggaggcgcccgcatgccatgcgcggagctcgtccgggagccgggctgcggctgctgctcggtgtgcgcccggctggagggcgaggcgtgcggcgtctacaccccgcgctgcggccaggggctgcgctgctatccccacccgggctccgagctgcccctgcaggcgctggtcatgggcgagggcacttgtgagaagcgccgggacgccgagtatggcgccagcccggagcaggttgcagacaatggcgatgaccactcagaaggaggcctggtggagaaccacgtggacagcaccatgaacatgttgggcgggggaggcagtgctggccggaagcccctcaagtcgggtatgaaggagctggccgtgttccgggagaaggtcactgagcagcaccggcagatgggcaagggtggcaagcatcaccttggcctggaggagcccaagaagctgcgaccaccccctgccaggactccctgccaacaggaactggaccaggtcctggagcggatctccaccatgcgccttccggatgagcggggccctctggagcacctctactccctgcacatccccaactgtgacaagcatggcctgtacaacctcaaacagtgcaagatgtctctgaacgggcagcgtggggagtgctggtgtgtgaaccccaacaccgggaagctgatccagggagcccccaccatccggggggaccccgagtgtcatctcttctacaatgagcagcaggaggcttgcggggtgcacacccagcggatgcagtagaccgcagccagccggtgcctggcgcccctgccccccgcccctctccaaacaccggcagaaaacggagagtgcttgggtggtgggtgctggaggattttccagttctgacacacgtatttatatttggaaagagaccagcaccgagctcggcacctccccggcctctctcttcccagctgcagatgccacacctgctccttcttgctttccccgggggaggaagggggttgtggtcggggagctggggtacaggtttggggagggggaagagaaatttttatttttgaacccctgtgtcccttttgcataagattaaaggaaggaaaagt

SEQ ID NO: 9 - protein, 328 aa, Homo sapiens
MLPRVGCPALPLPPPPLLPLLPLLLLLLGASGGGGGARAEVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPKKLRPPPARTPCQQELDQVLERISTMRLPDERGPLEHLYSLHIPNCDKHGLYNLKQCKMSLNGQRGECWCVNPNTGKLIQGAPTIRGDPECHLFYNEQQEACGVHTQRMQ

SEQ ID NO: 10 - DNA, 2506 bp, Homo sapiens
ggcacgaggcacagcttcgcgccgtgtactgtcgccccatccctgcgcgcccagcctgccaagcagcgtgccccggttgcaggcgtcatgcagcgggcgcgacccacgctctgggccgctgcgctgactctgctggtgctgctccgcgggccgccggtggcgcgggctggcgcgagctcggggggcttgggtcccgtggtgcgctgcgagccgtgcgacgcgcgtgcactggcccagtgcgcgcctccgcccgccgtgtgcgcggagctggtgcgcgagccgggctgcggctgctgcctgacgtgcgcactgagcgagggccagccgtgcggcatctacaccgagcgctgtggctccggccttcgctgccagccgtcgcccgacgaggcgcgaccgctgcaggcgctgctggacggccgcgggctctgcgtcaacgctagtgccgtcagccgcctgcgcgcctacctgctgccagcgccgccagctccaggaaatgctagtgagtcggaggaagaccgcagcgccggcagtgtggagagcccgtccgtctccagcacgcaccgggtgtctgatcccaagttccaccccctccattcaaagataatcatcatcaagaaagggcatgctaaagacagccagcgctacaaagttgactacgagtctcagagcacagatacccagaacttctcctccgagtccaagcgggagacagaatatggtccctgccgtagagaaatggaagacacactgaatcacctgaagttcctcaatgtgctgagtcccaggggtgtacacattcccaactgtgacaagaagggattttataagaaaaagcagtgtcgcccttccaaaggcaggaagcggggcttctgctggtgtgtggataagtatgggcagcctctcccaggctacaccaccaaggggaaggaggacgtgcactgctacagcatgcagagcaagtagacgcctgccgcaaggttaatgtggagctcaaatatgccttattttgcacaaaagactgccaaggacatgaccagcagctggctacagcctcgatttatatttctgtttgtggtgaactgattttttttaaaccaaagtttagaaagaggtttttgaaatgcctatggtttctttgaatggtaaacttgagcatcttttcactttccagtagtcagcaaagagcagtttgaattttcttgtcgcttcctatcaaaatattcagagactcgagcacagcacccagacttcatgcgcccgtggaatgctcaccacatgttggtcgaagcggccgaccactgactttgtgacttaggcggctgtgttgcctatgtagagaacacgcttcacccccactccccgtacagtgcgcacaggctttatcgagaataggaaaacctttaaaccccggtcatccggacatcccaacgcatgctcctggagctcacagccttctgtggtgtcatttctgaaacaagggcgtggatccctcaaccaagaagaatgtttatgtcttcaagtgacctgtactgcttggggactattggagaaaataaggtggagtcctacttgtttaaaaaatatgtatctaagaatgttctagggcactctgggaacctataaaggcaggtatttcgggccctcctcttcaggaatcttcctgaagacatggcccagtcgaaggcccaggatggcttttgctgcggccccgtggggtaggagggacagagagacagggagagtcagcctccacattcagaggcatcacaagtaatgtcacaattcttcggatgactgcagaaaatagtgttttgtagttcaacaactcaagacgaagcttatttctgaggataagctctttaaaggcaaagctttattttcatctctcatcttttgtcctccttagcacaatgtaaaaaagaatagtaatatcagaacaggaaggaggaatggcttgctggggagcccatccaggacactgggagcacatagagattcacccatgtttgttgaacttagagtcattctcatgcttttctttataattcacacatatatgcagagaagatatgttcttgttaacattgtatacaacatagccccaaatatagtaagatctatactagataatcctagatgaaatgttagagatgctatttgatacaactgtggccatgactgaggaaaggagctcacgcccagagactgggctgctctcccggaggccaaacccaagaaggtctggcaaagtcaggctcagggagactctgccctgctgcagacctcggtgtggacacacgctgcatagagctctccttgaaaacagaggggtctcaagacattctgcctacctattagcttttctttatttttttaactttttggggggaaaagtatttttgagaagtttgtcttgcaatgtatttataaatagtaaataaagtttttaccattaaaaaaataaaaaaaaaaaaaaaaaaaaaaaaaaaaaaaaaaaaaaaaaaaa

SEQ ID NO: 11 - protein, 291 aa, Homo sapiens
MQRARPTLWAAALTLLVLLRGPPVARAGASSGGLGPVVRCEPCDARALAQCAPPPAVCAELVREPGCGCCLTCALSEGQPCGIYTERCGSGLRCQPSPDEARPLQALLDGRGLCVNASAVSRLRAYLLPAPPAPGNASESEEDRSAGSVESPSVSSTHRVSDPKFHPLHSKIIIIKKGHAKDSQRYKVDYESQSTDTQNFSSESKRETEYGPCRREMEDTLNHLKFLNVLSPRGVHIPNCDKKGFYKKKQCRPSKGRKRGFCWCVDKYGQPLPGYTTKGKEDVHCYSMQSK

SEQ ID NO: 12 - DNA, 2160 bp, Homo sapiens
agccccctgcccctcgccgccccccgccgcctgcctgggccgggccgaggatgcggcgcagcgcctcggcggccaggcttgctcccctccggcacgcctgctaacttcccccgctacgtccccgttcgcccgccgggccgccccgtctccccgcggcctccgggtccgggtcctccaggacggccaggccgtgccgccgtgtgccctccgccgctcgcccgcgcgccgcgcgctccccgcctgcgcccagcgccccgcgcccgcgccccagtcctcgggcggtccatgctgcccctctgcctcgtggccgccctgctgctggccgccgggcccgggccgagcctgggcgacgaagccatccactgcccgccctgctccgaggagaagctggcgcgctgccgcccccccgtgggctgcgaggagctggtgcgagaggcgggctgcggctgttgcgccacttgcgccctgggcttggggatgccctgcggggtgtacaccccccgttgcggctcgggcctgcgctgctacccgccccgaggggtggagaagcccctgcacacactgatgcacgggcaaggcgtgtgcatggagctggcggagatcgaggccatccaggaaagcctgcagccctctgacaaggacgagggtgaccaccccaacaacagcttcagcccctgtagcgcccatgaccgcaggtgcctgcagaagcacttcgccaaaattcgagaccggagcaccagtgggggcaagatgaaggtcaatggggcgccccgggaggatgcccggcctgtgccccagggctcctgccagagcgagctgcaccgggcgctggagcggctggccgcttcacagagccgcacccacgaggacctctacttcatccccatccccaactgcgaccgcaacggcaacttccaccccaagcagtgtcacccagctctggatgggcagcgtggcaagtgctggtgtgtggaccggaagacgggggtgaagcttccggggggcctggagccaaagggggagctggactgccaccagctggctgacagctttcgagagtgaggcctgccagcaggccagggactcagcgtcccctgctactcctgtgctctggaggctgcagagctgacccagagtggagtctgagtctgagtcctgtctctgcctgcggcccagaagtttccctcaaatgcgcgtgtgcacgtgtgcgtgtgcgtgcgtgtgtgtgtgtttgtgagcatgggtgtgcccttggggtaagccagagcctggggtgttctctttggtgttacacagcccaagaggactgagactggcacttagcccaagaggtctgagccctggtgtgtttccagatcgatcctggattcactcactcactcattccttcactcatccagccacctaaaaacatttactgaccatgtactacgtgccagctctagttttcagccttgggaggttttattctgacttcctctgattttggcatgtggagacactcctataaggagagttcaagcctgtgggagtagaaaaatctcattcccagagtcagaggagaagagacatgtaccttgaccatcgtccttcctctcaagctagcccagagggtgggagcctaaggaagcgtggggtagcagatggagtaatggtcacgaggtccagacccactcccaaagctcagacttgccaggctccctttctcttcttccccaggtccttcctttaggtctggttgttgcaccatctgcttggttggctggcagctgagagccctgctgtgggagagcgaagggggtcaaaggaagacttgaagcacagagggctagggaggtggggtacatttctctgagcagtcagggtgggaagaaagaatgcaagagtggactgaatgtgcctaatggagaagacccacgtgctaggggatgaggggcttcctgggtcctgttcccctaccccatttgtggtcacagccatgaagtcaccgggatgaacctatccttccagtggctcgctccctgtagctctgcctccctctccatatctccttcccctacacctccctccccacacctccctactcccctgggcatcttctggcttgactggatggaaggagacttaggaacctaccagttggccatgatgtcttttctt

SEQ ID NO: 13 - protein, 258 aa, Homo sapiens
MLPLCLVAALLLAAGPGPSLGDEAIHCPPCSEEKLARCRPPVGCEELVREAGCGCCATCALGLGMPCGVYTPRCGSGLRCYPPRGVEKPLHTLMHGQGVCMELAEIEAIQESLQPSDKDEGDHPNNSFSPCSAHDRRCLQKHFAKIRDRSTSGGKMKVNGAPREDARPVPQGSCQSELHRALERLAASQSRTHEDLYFIPIPNCDRNGNFHPKQCHPALDGQRGKCWCVDRKTGVKLPGGLEPKGELDCHQLADSFRE

SEQ ID NO: 14 - DNA, 1722 bp, Homo sapiens
ggggaaaagagctaggaaagagctgcaaagcagtgtgggctttttccctttttttgctccttttcattacccctcctccgttttcacccttctccggacttcgcgtagaacctgcgaatttcgaagaggaggtggcaaagtgggagaaaagaggtgttagggtttggggtttttttgtttttgtttttgttttttaatttcttgatttcaacattttctcccaccctctcggctgcagccaacgcctcttacctgttctgcggcgccgcgcaccgctggcagctgagggttagaaagcggggtgtattttagattttaagcaaaaattttaaagataaatccatttttctctcccacccccaacgccatctccactgcatccgatctcattatttcggtggttgcttgggggtgaacaattttgtggctttttttcccctataattctgacccgctcaggcttgagggtttctccggcctccgctcactgcgtgcacctggcgctgccctgcttcccccaacctgttgcaaggctttaattcttgcaactgggacctgctcgcaggcaccccagccctccacctctctctacatttttgcaagtgtctgggggagggcacctgctctacctgccagaaattttaaaacaaaaacaaaaacaaaaaaatctccgggggccctcttggcccctttatccctgcactctcgctctcctgccccaccccgaggtaaagggggcgactaagagaagatggtgttgctcaccgcggtcctcctgctgctggccgcctatgcggggccggcccagagcctgggctccttcgtgcactgcgagccctgcgacgagaaagccctctccatgtgcccccccagccccctgggctgcgagctggtcaaggagccgggctgcggctgctgcatgacctgcgccctggccgaggggcagtcgtgcggcgtctacaccgagcgctgcgcccaggggctgcgctgcctcccccggcaggacgaggagaagccgctgcacgccctgctgcacggccgcggggtttgcctcaacgaaaagagctaccgcgagcaagtcaagatcgagagagactcccgtgagcacgaggagcccaccacctctgagatggccgaggagacctactcccccaagatcttccggcccaaacacacccgcatctccgagctgaaggctgaagcagtgaagaaggaccgcagaaagaagctgacccagtccaagtttgtcgggggagccgagaacactgcccacccccggatcatctctgcacctgagatgagacaggagtctgagcagggcccctgccgcagacacatggaggcttccctgcaggagctcaaagccagcccacgcatggtgccccgtgctgtgtacctgcccaattgtgaccgcaaaggattctacaagagaaagcagtgcaaaccttcccgtggccgcaagcgtggcatctgctggtgcgtggacaagtacgggatgaagctgccaggcatggagtacgttgacggggactttcagtgccacaccttcgacagcagcaacgttgagtgatgcgtccccccccaacctttccctcaccccctcccacccccagccccgactccagccagcgcctccctccaccccaggacgccactcatttcatctcatttaagggaaaaatatatatctatctatttgaggaaaaaaaaaaaaaaaaaaaa

SEQ ID NO: 15 - protein, 272 aa, Homo sapiens
MVLLTAVLLLLAAYAGPAQSLGSFVHCEPCDEKALSMCPPSPLGCELVKEPGCGCCMTCALAEGQSCGVYTERCAQGLRCLPRQDEEKPLHALLHGRGVCLNEKSYREQVKIERDSREHEEPTTSEMAEETYSPKIFRPKHTRISELKAEAVKKDRRKKLTQSKFVGGAENTAHPRIISAPEMRQESEQGPCRRHMEASLQELKASPRMVPRAVYLPNCDRKGFYKRKQCKPSRGRKRGICWCVDKYGMKLPGMEYVDGDFQCHTFDSSNVE

SEQ ID NO: 16 - DNA, 952 bp, Homo sapiens
gcagctgcgctgcgactgctctggaaggagaggacggggcacaaaccctgaccatgaccccccacaggctgctgccaccgctgctgctgctgctagctctgctgctcgctgccagcccaggaggcgccttggcgcggtgcccaggctgcgggcaaggggtgcaggcgggttgtccagggggctgcgtggaggaggaggatggggggtcgccagccgagggctgcgcggaagctgagggctgtctcaggagggaggggcaggagtgcggggtctacacccctaactgcgccccaggactgcagtgccatccgcccaaggacgacgaggcgcctttgcgggcgctgctgctcggccgaggccgctgccttccggcccgcgcgcctgctgttgcagaggagaatcctaaggagagtaaaccccaagcaggcactgcccgcccacaggatgtgaaccgcagagaccaacagaggaatccaggcacctctaccacgccctcccagcccaattctgcgggtgtccaagacactgagatgggcccatgccgtagacatctggactcagtgctgcagcaactccagactgaggtctaccgaggggctcaaacactctacgtgcccaattgtgaccatcgaggcttctaccggaagcggcagtgccgctcctcccaggggcagcgccgaggtccctgctggtgtgtggatcggatgggcaagtccctgccagggtctccagatggcaatggaagctcctcctgccccactgggagtagcggctaaagctgggggatagaggggctgcagggccactggaaggaacatggagctgtcatcactcaacaaaaaaccgaggccctcaatccaccttcaggccccgccccatgggcccctcaccgctggttggaaagagtgttggtgttggctggggtgtcaataaagctgtgcttggggtcaaa

SEQ ID NO: 17 - protein, 240 aa, Homo sapiens
MTPHRLLPPLLLLLALLLAASPGGALARCPGCGQGVQAGCPGGCVEEEDGGSPAEGCAEAEGCLRREGQECGVYTPNCAPGLQCHPPKDDEAPLRALLLGRGRCLPARAPAVAEENPKESKPQAGTARPQDVNRRDQQRNPGTSTTPSQPNSAGVQDTEMGPCRRHLDSVLQQLQTEVYRGAQTLYVPNCDHRGFYRKRQCRSSQGQRRGPCWCVDRMGKSLPGSPDGNGSSSCPTGSSG

SEQ ID NO: 18 - DNA, 1124 bp, Homo sapiens
gccgctgccaccgcaccccgccatggagcggccgtcgctgcgcgccctgctcctcggcgccgctgggctgctgctcctgctcctgcccctctcctcttcctcctcttcggacacctgcggcccctgcgagccggcctcctgcccgcccctgcccccgctgggctgcctgctgggcgagacccgcgacgcgtgcggctgctgccctatgtgcgcccgcggcgagggcgagccgtgcgggggtggcggcgccggcagggggtactgcgcgccgggcatggagtgcgtgaagagccgcaagaggcggaagggtaaagccggggcagcagccggcggtccgggtgtaagcggcgtgtgcgtgtgcaagagccgctacccggtgtgcggcagcgacggcaccacctacccgagcggctgccagctgcgcgccgccagccagagggccgagagccgcggggagaaggccatcacccaggtcagcaagggcacctgcgagcaaggtccttccatagtgacgccccccaaggacatctggaatgtcactggtgcccaggtgtacttgagctgtgaggtcatcggaatcccgacacctgtcctcatctggaacaaggtaaaaaggggtcactatggagttcaaaggacagaactcctgcctggtgaccgggacaacctggccattcagacccggggtggcccagaaaagcatgaagtaactggctgggtgctggtatctcctctaagtaaggaagatgctggagaatatgagtgccatgcatccaattcccaaggacaggcttcagcatcagcaaaaattacagtggttgatgccttacatgaaataccagtgaaaaaaggtgaaggtgccgagctataaacctccagaatattattagtctgcatggttaaaagtagtcatggataactacattacctgttcttgcctaataagtttcttttaatccaatccactaacactttagttatattcactggttttacacagagaaatacaaaataaagatcacacatcaagactatctacaaaaatttattatatatttacagaagaaaagcatgcatatcattaaacaaataaaatactttttatcacaaaaaaaaaaaaaaaa

SEQ ID NO: 19 - protein, 282 aa, Homo sapiens
MERPSLRALLLGAAGLLLLLLPLSSSSSSDTCGPCEPASCPPLPPLGCLLGETRDACGCCPMCARGEGEPCGGGGAGRGYCAPGMECVKSRKRRKGKAGAAAGGPGVSGVCVCKSRYPVCGSDGTTYPSGCQLRAASQRAESRGEKAITQVSKGTCEQGPSIVTPPKDIWNVTGAQVYLSCEVIGIPTPVLIWNKVKRGHYGVQRTELLPGDRDNLAIQTRGGPEKHEVTGWVLVSPLSKEDAGEYECHASNSQGQASASAKITVVDALHEIPVKKGEGAEL

SEQ ID NO: 20 - DNA, 2125 bp, Homo sapiens
ggcacagcagacgtaccctccctcgctgcctgcctgcggcctgccctgcatgcaggatggccctgaggaaaggaggcctggccctggcgctgctgctgctgtcctgggtggcactgggcccccgcagcctggagggagcagaccccggaacgccgggggaagccgagggcccagcgtgcccggccgcctgtgtctgcagctacgatgacgacgcggatgagctcagcgtcttctgcagctccaggaacctcacgcgcctgcctgacggagtcccgggcggcacccaagccctgtggctggacggcaacaacctctcgtccgtccccccggcagccttccagaacctctccagcctgggcttcctcaacctgcagggcggccagctgggcagcctggagccacaggcgctgctgggcctagagaacctgtgccacctgcacctggagcggaaccagctgcgcagcctggcactcggcacgtttgcacacacgcccgcgctggcctcgctcggcctcagcaacaaccgtctgagcaggctggaggacgggctcttcgagggcctcggcagcctctgggacctcaacctcggctggaatagcctggcggtgctccccgatgcggcgttccgcggcctgggcagcctgcgcgagctggtgctggcgggcaacaggctggcctacctgcagcccgcgctcttcagcggcctggccgagctccgggagctggacctgagcaggaacgcgctgcgggccatcaaggcaaacgtgttcgtgcagctgccccggctccagaaactctacctggaccgcaacctcatcgctgccgtggccccgggcgccttcctgggcctgaaggcgctgcgatggctggacctgtcccacaaccgcgtggctggcctcctggaggacacgttccccggtctgctgggcctgcgtgtgctgcggctgtcccacaacgccatcgccagcctgcggccccgcaccttcaaggacctgcacttcctggaggagctgcagctgggccacaaccgcatccggcagctggctgagcgcagctttgagggcctggggcagcttgaggtgctcacgctagaccacaaccagctccaggaggtcaaggcgggcgctttcctcggcctcaccaacgtggcggtcatgaacctctctgggaactgtctccggaaccttccggagcaggtgttccggggcctgggcaagctgcacagcctgcacctggagggcagctgcctgggacgcatccgcccgcacaccttcaccggcctctcggggctccgccgactcttcctcaaggacaacggcctcgtgggcattgaggagcagagcctgtgggggctggcggagctgctggagctcgacctgacctccaaccagctcacgcacctgccccaccgcctcttccagggcctgggcaagctggagtacctgctgctctcccgcaaccgcctggcagagctgccggcggacgccctgggccccctgcagcgggccttctggctggacgtctcgcacaaccgcctggaggcattgcccaacagcctcttggcaccactggggcggctgcgctacctcagcctcaggaacaactcactgcggaccttcacgccgcagcccccgggcctggagcgcctgtggctggagggtaacccctgggactgtggctgccctctcaaggcgctgcgggacttcgccctgcagaaccccagtgctgtgccccgcttcgtccaggccatctgtgagggggacgattgccagccgcccgcgtacacctacaacaacatcacctgtgccagcccgcccgaggtcgtggggctcgacctgcgggacctcagcgaggcccactttgctccctgctgaccaggtccccggactcaagccccggactcaggcccccacctggctcaccttgtgctggggacaggtcctcagtgtcctcaggggcctgcccagtgcacttgctggaagacgcaagggcctgatggggtggaaggcatggcggcccccccagctgtcatcaattaaaggcaaaggcaatcgaaaaaaaaaaaaaaaaaaaaaaaaaaaaaaaaaaaaaaaaaaaaaaaaaaaaaaaaaaaaaaaaaaaaaaa

SEQ ID NO: 21 - protein, 605 aa, Homo sapiens
MALRKGGLALALLLLSWVALGPRSLEGADPGTPGEAEGPACPAACVCSYDDDADELSVFCSSRNLTRLPDGVPGGTQALWLDGNNLSSVPPAAFQNLSSLGFLNLQGGQLGSLEPQALLGLENLCHLHLERNQLRSLALGTFAHTPALASLGLSNNRLSRLEDGLFEGLGSLWDLNLGWNSLAVLPDAAFRGLGSLRELVLAGNRLAYLQPALFSGLAELRELDLSRNALRAIKANVFVQLPRLQKLYLDRNLIAAVAPGAFLGLKALRWLDLSHNRVAGLLEDTFPGLLGLRVLRLSHNAIASLRPRTFKDLHFLEELQLGHNRIRQLAERSFEGLGQLEVLTLDHNQLQEVKAGAFLGLTNMAVMNLSGNCLRNLPEQVFRGLGKLHSLHLEGSCLGRIRPHTFTGLSGLRRLFLKDNGLVGIEEQSLWGLAELLELDLTSNQLTHLPHRLFQGLGKLEYLLLSRNRLAELPADALGPLQRAFWLDVSHNRLEALPNSLLAPLGRLRYLSLRNNSLRTFTPQPPGLERLWLEGNPWDCGCPLKALRDFALQNPSAVPRFVQAICEGDDCQPPAYTYNNITCASPPEVVGLDLRDLSEAHFAPC